(19) United States
(12) Patent Application Publication
(10) Pub. No.: US 2023/0097658 A1
(43) Pub. Date: Mar. 30, 2023

(54) TUNABLE EXTENDED RELEASE HYDROGELS

(71) Applicants: FRED HUTCHINSON CANCER RESEARCH CENTER, Seattle, WA (US); UNIVERSITY OF WASHINGTON, Seattle, WA (US)

(72) Inventors: James Olson, Seattle, WA (US); Eric Nealy, Seattle, WA (US); Cole DeForest, Seattle, WA (US); Andrew Mhyre, Seattle, WA (US)

(73) Assignees: FRED HUTCHINSON CANCER RESEARCH CENTER, Seattle, WA (US); UNIVERSITY OF WASHINGTON, Seattle, WA (US)

(21) Appl. No.: 17/905,011

(22) PCT Filed: Feb. 26, 2021
(86) PCT No.: PCT/US2021/019937

§ 371 Date: Aug. 25, 2022

Related U.S. Application Data
(60) Provisional application No. 62/982,609, filed on Feb. 27, 2020.

(51) Int. Cl. C07K 16/28; A61K 9/00; A61P 35/00

(52) U.S. Cl. CPC C07K 16/2803; A61K 9/0024; A61P 35/00; A61K 2039/505

(57) ABSTRACT

The present disclosure describes an immunotherapy delivery hydrogel system. The immunotherapy delivery hydrogel system can be degradable and can release therapeutic agents at a tunable rate, and in a controlled manner. The immunotherapy delivery hydrogel system includes a hydrogel matrix and cancer therapeutic agent(s) associated with the hydrogel matrix. The hydrogel system can further include tumor cell-attractant(s) conjugated to the hydrogel matrix. The tumor cell-attractant(s) and the cancer therapeutic agent(s) act synergistically to treat cancer.

### CROSS-REFERENCE(S) TO RELATED APPLICATION(S)

This application claims the benefit of U.S. Patent Application No. 62/982,609, filed Feb. 27, 2020, the disclosure of which is incorporated herein by reference in its entirety.

### STATEMENT OF GOVERNMENT LICENSE RIGHTS

This invention was made with government support under CA080416 awarded by the National Institutes of Health. The government has certain rights in this invention.

### STATEMENT REGARDING SEQUENCE LISTING

The sequence listing associated with this application is provided in text format in lieu of a paper copy and is hereby incorporated by reference into the specification. The name of the text file containing the sequence listing is FHTM173667_SEQ_final_20210225_ST25.txt. The text file is 22 KB; was created on Feb. 24, 2021; and is being submitted with the filing of the specification.

### BACKGROUND

Biomaterials are implantable substances intended to coexist within biological settings and can have diverse applications. Biomaterials can include metallic, alloy, polymeric, or ceramic origins. For use with biological systems, materials should have a minimal risk of chronic inflammation and of mechanically harming sensitive tissue.

### Hydrogels

Hydrogels are crosslinked polymers that possess the ability to swell upwards of 90% their total volume with fluid, allowing for the diffusion of nutrients and molecules between the tissue and the gel itself. Hydrogels can be used as biomaterials and can be categorized according to whether they are of natural or synthetic origin. Natural hydrogels are composed primarily of biomolecules such as proteins and polysaccharides (e.g., fibrin, chitosan, and hyaluronan). Some advantages of this type of gel include the ability to perform biological functions such as cell adhesion and degradation. However, natural hydrogels can have poor mechanical properties and high immunogenic activity. In contrast, synthetic hydrogels possess properties that can make them suitable in drug delivery applications.

Using hydrogels as drug delivery devices can present certain challenges, such as a limited area of effect, because therapeutic agents delivered locally from a static source can only have a small area where they are present at sufficiently high concentrations. For example, a chemotherapeutic agent can physically reach migratory tumor cells only a few centimeters away, but at insufficient concentrations to cause cell death. Similarly, delivery of blocking antibodies to macrophage checkpoints at sufficient antibody concentrations to enact a phagocytic response on tumor cells can only exist at close proximity to a static source of therapeutic agent.

Chemokines and their Receptors

Chemokines are soluble cytokines that elicit directed migration patterns of a cells and organisms—a process known as chemotaxis. There are over 50 different types of chemokines conserved between humans and mice. Chemokines can be between 7-15 kDa in molecular weight. Chemokines are grouped into sub-divisions based on the position of the first cysteine amino acids within their primary structure. Thus, chemokines fall into the C, CC, CX3C and CXC varieties. Physiologically, chemokines play a number of roles in mammals, including: guiding cells through the developing embryo, axon motility, wound healing, and immune cells trafficking. Chemokines are physiologically secreted by a wide variety of cell types in the body, including astrocytes, immune cells, and endothelial cells. Once secreted, chemokines can form physical and chemical gradients for cells to follow, either through binding to glycosaminoglycans on cell surfaces or dispersing through the fluids of the extracellular space. Cell types responsive to these chemical cues express chemokine receptors and travel up the concentration gradient of a particular chemokine to their destination.

There are approximately 25 chemokine receptors responsible for chemokine binding. 20 receptors are G-protein coupled receptors (GPCRs) and are for downstream signaling responses. The remaining 5 chemokine receptors are non-signaling and are referred to as “Atypical”. While GPCRs can induce movement of a particular cell type, atypical chemokine receptors can be used for chemokine scavenging from the microenvironment to prevent GPCR over-saturation. The structure of the GPCR has 7 transmembrane domains, a glycosylated N-terminus and a phosphorylated C terminus that is used for recruitment of a family of proteins called arrestins. Without wishing to be bound by theory, it is believed that chemokines bind to their receptors through a two-step, two-site process, where each site individually imparts specificity and receptor activation. The main body of each chemokine binds to the N-terminus and transmembrane loops of the chemokine receptor. These interactions are specific for each family of receptor. The N-terminus of the chemokine then binds inside of the transmembrane domain on the receptor, activating downstream signaling.

Ligand binding to the chemokine receptor is the first of four major events leading to chemotaxis. The other three are signal transduction from the GPCR. cytoskeleton re-arrangement, and the establishment of polarity of the cell. Transduction of the signal is a central figure in this process as it receives and relays input from the G-protein network, cytoskeletal and polarity biochemical pathways. These factors work in concert to ultimately result in the cell's cytoskeleton forming pseudopodia in the direction of the gradient, the leading edge, and contraction of the cytoskeleton at the lagging edge.

In addition to the many physiological roles of chemotaxis (e.g., immune cell trafficking, embryonic development, etc.), it is also believed to play a major role in the metastasis of various cancer types. Each of the diverse types of metastatic cancers originating from a particular organ system tends to spread to the same secondary locations in the body, regardless of the patient. For example, metastatic breast cancer tends to spread to the bones, liver, the brain, and the lungs. Without wishing to be bound by theory, it is believed that these tumor cells can home in on chemokine gradients expressed endogenously at these sites. Over 20 different cancer types are known to express chemokine receptors at levels higher than those found in the surrounding healthy parenchyma, and it is believed that this increased receptor expression may play a contributing role in the “homing” properties these metastatic cancers have for their respective niches. As an example, breast tumor cells often over-express CXCR4, the receptor for CXCL12. This chemokine is naturally secreted in areas of the body where this tumor type tends to metastasize: brain, lungs, lymph nodes and bones.

As another example, pediatric brain tumors (PBTs) are the most commonly diagnosed solid tumor in children. These malignancies rank among the leading causes of pediatric cancer-related death, rivaled only by leukemia. Standard of care surgery and radiation for these tumors are often complicated by the location of tumor onset because the majority of PBTs manifest in areas of the brain where complete tumor removal and radiation therapy could permanently impair a patient's cognitive, behavioral, and motor functions. Surgeons often have to choose whether to leave tumor tissue behind or risk taking too much healthy tissue leading to life-long adverse effects. However, residual brain tumor cells can regrow and metastasize, ultimately leading to patient death.

Many systemically administered immunotherapeutic approaches utilized for adults are unlikely to be successful for PBTs. These tumors have notoriously low mutation burdens and often do not express tumor-specific markers to target with T-cell mediated approaches. Furthermore, trafficking of immune cells across the BBB is tightly regulated. As for the efficacy of locally delivered immuno-stimulatory molecules within the perioperative cavity of incompletely removed brain tumors, the approaches often utilize bulky osmotic pumps and biomaterials that either protrude from the skull or do not afford prolonged release rates of their payloads, possibly necessitating multiple surgeries when translated to human patients. Furthermore, tumor cells that migrate more than a few millimeters away from the tumor cavity may be out of reach from an implanted drug depot due to poor diffusion of therapeutics through the brain's parenchyma.

Brain tumor cells, of both adult and pediatric origins, also tend to over-express chemokine receptors whose natural ligands are found within known patterns throughout the brain. In many cases, without wishing to be bound by theory, the chemokine's most prominent effects on brain tumor cells appear to be both a migration signal and a mitogen. As such, many pre-clinical therapeutic strategies targeting the activity of chemokine receptors in brain tumor cells are designed to block their function. For example, CTCE-9908 is a competitive inhibitor of CXCR4 with reduced agonist and signaling capabilities compared to CXCL12. This drug was shown in a mouse model of osteosarcoma to reduce tumor cell growth and adhesion, but most importantly reduced metastatic spread. AMD3100 is another inhibitor of CXCR4 that has been investigated against a variety of tumor types, including brain. In one study, blocking this receptor demonstrated reduced chemotaxis of a human GB line towards gradients of CXCL12.

There is a need for a biocompatible, tunable hydrogel-based therapeutic agent delivery system that allows for extended release of pharmaceutical agents such as proteins and monoclonal antibodies. The present disclosure fulfills these needs and presents further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features an immunotherapy delivery hydrogel system, including a hydrogel matrix; a tumor cell-attractant conjugated to the hydrogel matrix; and a cancer therapeutic agent associated with the hydrogel matrix. The tumor cell-attractant and the cancer therapeutic agent can be synergistic in treating cancer and are controllably released from the immunotherapy delivery hydrogel system.

In another aspect, the present disclosure features a method of treating cancer, including administrating an immunotherapy delivery hydrogel system described herein to a subject in need thereof. The cancer can have an upregulation of PD-L1, CTLA-4, CD47, CD24, CD155, CD112, β₂ Microglobulin (B2M), or any combination thereof.

In yet another aspect, the present disclosure features a method of making a recombinant protein, including: providing a gene fragment for a protein sequence comprising an enzyme-recognizable label; inserting the gene fragment into a plasmid; transducing the plasmid into a mammalian cell; expressing a protein encoded by the gene fragment; and isolating the protein.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic representation of certain exemplary poly(ethylene glycol) PEG chains as backbones for hydrogel formation. PEG has low inherent immunogenicity and can be functionalized at one or multiple arms with chemical handles to make it amenable to bio-orthogonal chemistries and polymerization strategies.

FIG. 2 shows the sequences of mCXCL12 expression constructs for E. coli.

FIG. 3A is a series of photographs of embodiments of hydrogel matrices of the present disclosure, as a function of the amounts of hydrolysable 2-azidoester crosslinker, measured at 0 hour, 24 hours, and 96 hours.

FIG. 3B is a graph of a release of fluorescent AF568 dye covalently-conjugated to embodiments of hydrogel matrices of the present disclosure, as a function of the amounts of hydrolysable 2-azidoester crosslinker.

FIGS. 4A-4D demonstrate that human pHGG cells are sensitive to chemokines.

FIG. 4A is an immunohistochemistry (IHC) image of xenografted PBT-05, a patient derived pediatric high-grade glioma (pHGG). This tumor type is highly infiltrative in the brains of a mouse model as it was in human patients.

FIG. 4B is a series of chemotaxis assays using PBT05, demonstrating varying sensitivity to the classical chemokines: CCL2 and CXCL12. Human variants were more potent than their murine homologs.

FIG. 4C shows photographs of GFP+ PBT05 cells seeded on a transwell, exposed to chemoattractants diffusing from a lower chamber. After 96 hours (right image), many cells seeded on top migrated to the bottom, where the Incucyte software masks them as red.

FIG. 4D shows the flow cytometry data for the canonical receptors of CCL2 and CXCL12, revealing no expression of CCR2, but high levels of CXCR4, respectively.

FIGS. 5A and 5B demonstrate that the blockade of macrophage checkpoints on pHGG cells was an effective strategy to induce phagocytosis by murine and human macrophages.

FIG. 5A is a series of flow cytometry plots of PBT05, showing high levels of the macrophage checkpoint, CD47, and moderate expression of CD24, another macrophage checkpoint.

FIG. 5B is a series of graphs showing the relative number of tumor cells in PBT05 cells co-cultured with murine bone marrow derived macrophages (BMDM) or human macrophages derived from PBMCs. Co-cultures or PBT05 alone were challenged with various immunomodulators. CD47 mAb blockade was the most effective single agent in both groups, though to a higher degree with murine macrophages. Combinations of CD47mAb and CD24mAb elicited higher degrees of phagocytosis in human macrophage co-cultures than CD47mAb alone. No toxicity was observed in tumor cells cultured alone.

FIGS. 6A-6C are directed to the synthesis of a highly customizable hydrogel to serve as an in vivo delivery depot.

FIG. 6A is a schematic summarizing payload release from a PEG-tetraBCN hydrogel into release media. Hydrolysable azidoesters are employed as linkers to tune molecule release rates from the gel.

FIG. 6B is a series of graphs quantifying the release of a small fluorescent molecule, coumarin, over 4 weeks using various azidoester linkers. The 4-carbon linker was chosen for further experiments and significantly slowed release of coumarin compared to diffusion alone.

FIG. 6C is a hematoxylin and eosin (H&E) stain of a murine brain containing a polymerized PEG-tetraBCN gel after being injected into the parenchyma.

FIGS. 7A-7D are directed to sortagging CD47mAbs and CXCL12 to hydrolysable azidoester linkers. Biomolecules of interest can be site-specifically sortagged to azidoester linkers for extended release while retaining biological activity.

FIG. 7A is a schematic representation of a Steglich esterification, where the prerequisite GGG-containing polypeptide needed for sortase tagging was conjugated to the azidoacid linker.

FIG. 7B is a schematic representation of recombinantly expressed CXCL12 and CD47mAbs produced with LPETG sortase recognition sites at their C-termini. Running the sortase reaction with these molecules and the polypeptide-azidoacid conjugate C-terminally labels the biomolecule of interest with a hydrolysable linker, allowing conjugation to gels via SPAAC click chemistry.

FIG. 7C shows the sequences of MCXCL12 expression constructs.

FIG. 7D shows the sequences of CD47mAb expression constructs.

FIGS. 8A-8B are directed to locally released mCXCL12 and CD47mAb, which recruit pHGG cells into an immunotherapy trap in vivo.

FIG. 8A is a schematic representation of an embodiment of an immunotherapy trap within a mouse model. Gels only delivering blocking antibodies to macrophage checkpoints can be effective at promoting phagocytosis of tumor cells in close range but can be ineffective at eliminating cells that have migrated a sufficient distance away from the gel implant. Gels that contain a chemokine lure can attract distant tumor cells closer to effective concentrations of therapeutics they would otherwise avoid.

FIG. 8B is a graph quantifying differences in tumor bioluminescence of mice receiving gels from different treatment groups. Gels treated with CD47mAb in combination with CXCL12 show arrested tumor growth over the course of two weeks. All other conditions continued growing at the same pace as mice receiving untreated gels.

### DETAILED DESCRIPTION

The present disclosure describes an immunotherapy delivery hydrogel system. The immunotherapy delivery hydrogel system can be degradable and can release therapeutic agents at a tunable rate and in a controlled manner. The immunotherapy delivery hydrogel system includes a hydrogel matrix and cancer therapeutic agent(s) associated with the hydrogel matrix. The hydrogel system can further include tumor cell-attractant(s) conjugated to the hydrogel matrix. The tumor cell-attractant(s) and the cancer therapeutic agent(s) are synergistic in treating cancer and are controllably released independently from the hydrogel.

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a saturated hydrocarbon group which is straight-chained (e.g., linear) or branched. Example alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. An alkyl group can contain from 1 to about 30, from 1 to about 24, from 2 to about 24, from 1 to about 20, from 2 to about 20, from 1 to about 10, from 1 to about 8, from 1 to about 6, from 1 to about 4, or from 1 to about 3 carbon atoms.

As used herein, the term “aryl” refers to monocyclic or polycyclic (e.g., having 2, 3, or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “alkylene” refers to a linking alkyl group.

As used herein, “alkenyl” refers to an alkyl group having one or more double carbon-carbon bonds. The alkenyl group can be linear or branched. Example alkenyl groups include ethenyl, propenyl, and the like. An alkenyl group can contain from 2 to about 30, from 2 to about 24, from 2 to about 20, from 2 to about 10, from 2 to about 8, from 2 to about 6, or from 2 to about 4 carbon atoms.

As used herein, “alkenylene” refers to a linking alkenyl group.

As used herein, “alkynyl” refers to an alkyl group having one or more triple carbon-carbon bonds. The alkynyl group can be linear or branched. Example alkynyl groups include ethynyl, propynyl, and the like. An alkynyl group can contain from 2 to about 30, from 2 to about 24, from 2 to about 20, from 2 to about 10, from 2 to about 8, from 2 to about 6, or from 2 to about 4 carbon atoms.

As used herein, “alkynylene” refers to a linking alkynyl group.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “haloalkenyl” refers to an alkenyl group having one or more halogen substituents.

As used herein, “haloalkynyl” refers to an alkynyl group having one or more halogen substituents.

As used herein, “haloalkoxy” refers to an —O-(haloalkyl) group.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “arylene” refers to a linking aryl group.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached though either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcamyl, adamantyl, and the like.

As used herein, “cycloalkylene” refers to a linking cycloalkyl group.

As used herein, “heteroalkyl” refers to an alkyl group having at least one heteroatom such as sulfur, oxygen, or nitrogen.

As used herein, “heteroalkylene” refers to a linking heteroalkyl group.

As used herein, a “heteroaryl” refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heteroarylene” refers to a linking heteroaryl group.

As used herein, “amino” refers to NH₂.

As used herein, “alkylamino” refers to an amino group substituted by an alkyl group.

As used herein, “dialkylamino” refers to an amino group substituted by two alkyl groups.

As used herein, the term “random copolymer” is a copolymer having an uncontrolled mixture of two or more constitutional units. The distribution of the constitutional units throughout a polymer backbone can be a statistical distribution, or approach a statistical distribution, of the constitutional units. In some embodiments, the distribution of one or more of the constitutional units is favored. For a polymer made via a controlled polymerization (e.g., RAFT, ATRP, ionic polymerization), a gradient can occur in the polymer chain, where the beginning of the polymer chain (in the direction of growth) can be relatively rich in a constitutional unit formed from a more reactive monomer while the later part of the polymer can be relatively rich in a constitutional unit formed from a less reactive monomer, as the more reactive monomer is depleted. To decrease differences in distribution of the constitutional units, comonomers in the same family (e.g., methacrylate-methacrylate, acrylamide-acrylamido) can be used in the polymerization process, such that the monomer reactivity ratios are similar.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety that is not attracted to water with significant apolar surface area at physiological pH and/or salt conditions. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar functional groups such as amides, hydroxyl groups, or ethylene glycol residues. Examples of hydrophilic moieties include carboxylic acid groups, amino groups, hydroxyl groups, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “peptide” refers to natural biological or artificially manufactured short chains of amino acid monomers linked by peptide (amide) bonds. As used herein, a peptide has at least 2 amino acid repeating units.

As used herein, the term “oligomer” refers to a macromolecule having 10 or less repeating units.

As used herein, the term “polymer backbone” or “backbone” refers to that portion of the polymer which is a continuous chain, including the bonds which are formed between monomers upon polymerization. The composition of the polymer backbone can be described in terms of the identity of the monomers from which it is formed, without regard to the composition of branches, or side chains, off of the polymer backbone. Thus, poly(acrylic acid) is said to have a substituted poly(ethylene) backbone with carboxylic acid (—CO)OH) groups as side chains.

As used herein, the term “polymer” refers to a macromolecule having more than 10 repeating units.

As used herein, the term “polysaccharide” refers to a carbohydrate that can be decomposed by hydrolysis into two or more molecules of monosaccharides.

As used herein, the term “hydrogel” refers to a water-swollen, and cross-linked polymeric network produced by the reaction of one or more monomers. The polymeric material exhibits the ability to swell and retain a significant fraction of water within its structure but does not dissolve in water.

As used herein, the term “protein” refers to any of various naturally occurring extremely complex substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

As used herein, the term “tissue” refers to an aggregate of similar cells and cell products forming a definite kind of structural material with a specific function, in a multicellular organism.

As used herein, the term “organs” refers to a group of tissues in a living organism that have been adapted to perform a specific function.

As used herein, the term “therapeutic agent” refers to a substance capable of producing a curative effect in a disease state.

As used herein, the term “small molecule” refers to a low molecular weight (<2000 daltons) organic compound that may help regulate a biological process, with a size on the order of 1 nm.

As used herein, the term “antibody” refers to an intact immunoglobulin including monoclonal antibodies, or to an antigen-binding and/or variable domain comprising fragment of an immunoglobulin that competes with the intact immunoglobulin for specific binding to the binding partner of the immunoglobulin, i.e., regardless of structure, the antigen-binding fragment binds with the same antigen that is recognized by the intact immunoglobulin. An antigen-binding fragment can comprise a peptide or polypeptide comprising an amino acid sequence of at least 2, 5, 10, 15, 20, 25, 30, 35, 40, 50, 60, 70, 80, 90, 100, 125, 150, 175, 200, or 250 contiguous amino acid residues of the amino acid sequence of the binding molecule. The term “antibody”, as used herein includes all immunoglobulin classes and subclasses known in the art. Depending on the amino acid sequence of the constant domain of their heavy chains, antibodies can be divided into the five major classes of intact antibodies: IgA, IgD, IgE, IgG, and IgM, and several of these may be further divided into subclasses (isotypes), e.g., IgA1, IgA2, IgG1, IgG2, IgG3 and IgG4.

As used herein, the term “monoclonal antibody” refers to a preparation of antibody molecules of single specificity. A monoclonal antibody displays a single binding specificity and affinity for a particular epitope. Accordingly, the term “human monoclonal antibody” refers to an antibody displaying a single binding specificity which has variable and constant regions derived from or based on human germline immunoglobulin sequences or derived from completely synthetic sequences. The method of preparing the monoclonal antibody is not relevant for the binding specificity.

Antigen-binding fragments include, inter alia, Fab, F(ab′), F(ab′)2, Fv, dAb, Fd, complementarity determining region (CDR) fragments, single-chain antibodies (scFv), bivalent single-chain antibodies, single-chain phage antibodies, diabodies, triabodies, tetrabodies, (poly)peptides that contain at least a fragment of an immunoglobulin that is sufficient to confer specific antigen binding to the polypeptide, etc. The above fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, Edited by: E. Harlow and D, Lane (1988), Cold Spring Harbor Laboratory, Cold Spring Harbor, N.Y.

As used herein, the term “biomaterial” refers to a natural or synthetic material (such as a metal or polymer) that is suitable for introduction into living tissue, for example, as part of a medical device (such as an artificial joint).

As used herein, the term “ceramic” refers to an inorganic, non-metallic, solid material comprising metal, non-metal or metalloid atoms primarily held in ionic and covalent bonds.

As used herein, the term “composite” refers to a composition material, a material made from two or more constituent materials with significantly different physical or chemical properties that, when combined, produce a material with characteristics different from the individual components. The individual components remain separate and distinct within the finished structure.

One letter codes for amino acids are used herein. For example, alanine is A, arginine is R, asparagine is N, aspartic acid is D, asparagine or aspartic acid is B, cysteine is C, glutamic acid is E, glutamine is Q, glutamine or glutamic acid is Z, glycine is G, histidine is H, isoleucine is I, leucine is L, lysine is K, methionine is M, phenylalanine is F, proline is P, serine is S, threonine is T, tryptophan is W, tyrosine is Y, valine is V.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means +/−5%. As used herein, recited ranges include the end points, such that from 0.5 mole percent to 99.5 mole percent includes both 0.5 mole percent and 99.5 mole percent.

### Immunotherapy Delivery Hydrogel System

As described above, the immunotherapy delivery hydrogel system includes a hydrogel matrix and cancer therapeutic agent(s) associated with the hydrogel matrix. The immunotherapy delivery hydrogel system can further include tumor cell-attractant(s) conjugated to the hydrogel matrix. The tumor cell-attractant(s) and the cancer therapeutic agent(s) are controllably released from the hydrogel, and can act synergistically in treating cancer, such that the tumor cell-attractant(s) and the cancer therapeutic agent(s), acting together, have greater effectiveness in treating the cancer than the added effects of each of the tumor cell-attractant(s) and the cancer therapeutic agent(s), acting separately. The immunotherapy delivery hydrogel system can allow for extended release of therapeutic agent(s), such as proteins and monoclonal antibodies.

Localized, slow release of therapeutic agents into or around the tumor bed has a number of advantages. For example, higher concentrations of active agents can be delivered to the tumor site than can be achieved systemically. The slow release aspect of the implanted biomaterial can attenuate toxicities related to burst release of drugs into tissue.

Hydrogel Matrix

The hydrogel matrix can be formed of a multivalent polymer building block (also referred to herein as a hydrogel precursor) and a crosslinker to crosslink the multivalent polymer building block. The hydrogel matrix can be degradable. The tumor cell-attractant and the cancer therapeutic agent can each independently have one or more reactive groups that react with the multivalent polymer building blocks, the crosslinker, and/or the hydrogel matrix. In some embodiments, the tumor cell-attractant and the cancer therapeutic agent can each independently be covalently attached to the hydrogel matrix by a linker. The linker can include a cleavable group.

Suitable hydrogel precursors include monomers and macromers. As used herein, the term “hydrogel precursor(s)” or “precursor compound” or “multivalent polymer building blocks” in the context of the hydrogel matrix refers to components that can be reacted to form a hydrogel, either with or without the use of an initiator. As used herein, the terms “reactive precursor(s)” include precursors that can crosslink upon exposure to each other to form a hydrogel. As used herein, the term “initiated precursor(s)” refers to hydrogel precursors that crosslink upon exposure to an external source, sometimes referred to herein as an “initiator.” Initiators include, for example, radicals, ions, UV light, redox-reaction components, and combinations thereof, as well as other initiators within the purview of those skilled in the art.

The hydrogel matrix can be formed of a network of polymeric chains of molecules. In some embodiments, the polymeric chains can be synthetic. These gels can be polymerized using a wide variety of techniques such as photopolymerization, enzymatic reactions and bio-orthogonal click chemistry. Synthetic hydrogels can contain adjustable mechanical properties and stiffness, allowing the user to tune the physical stress the gels impart on the surrounding tissue.

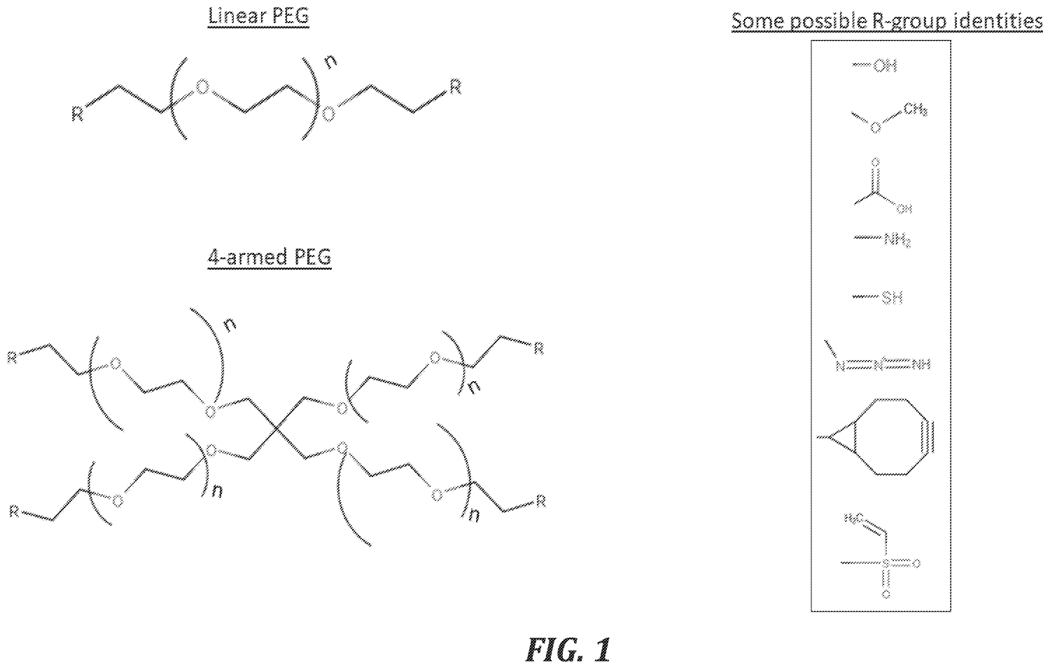

The hydrogel precursors can include biologically inert and/or water-soluble polymer regions. When the polymeric region is water soluble, suitable polymers include polyethers, for example, polyalkylene oxides such as polyethylene glycol (“PEG”), polyethylene oxide (“PEO”), polyethylene oxide-co-polypropylene oxide (“PPO”), co-polyethylene oxide block or random copolymers, polyacrylamide (PAAm), polyvinyl alcohol (“PVA”); poly(vinyl pyrrolidinone) (“PVP”); poly(amino acids); poly(saccharides), such as dextran, chitosan, alginates, carboxymethylcellulose, oxidized cellulose, hydroxyethylcellulose and/or hydroxymethylcellulose; hyaluronic acid; and proteins such as albumin, collagen, casein, and gelatin. In some embodiments, combinations of the above-described polymeric materials can form the hydrogel matrix. The polyethers, and more particularly poly(oxyalkylenes) or poly(ethylene glycol) or polyethylene glycol (“PEG”), can form the hydrogel matrix in some embodiments. Non limiting examples of PEG multivalent polymer building blocks and crosslinkers are shown in FIG. 1.

In some embodiments, the hydrogel matrix are tissue-integrating hydrogels, for example, the hydrogel matrix can include materials disclosed in U.S. Pat. No. 10,117,613, the disclosure of which is incorporated herein by reference in its entirety.

The hydrogel matrix can include a crosslinked polymer, such as a crosslinked poly(ethylene glycol). The hydrogel matrix can be formed of multivalent polymer building blocks and a crosslinker. The multivalent polymer building blocks can include oligomeric building blocks, and can be linear, branched, a star polymer, and/or a dendritic polymer. The multivalent polymer building blocks can include a plurality of reactive groups orthogonal to reactive groups present in the tumor cell-attractant and the cancer therapeutic agent. For example, the multivalent polymer building blocks can include SH, OH, amino, COOH, ester (e.g., an activated ester), N₃, optionally substituted maleimide, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive groups. In some embodiments, the reactive groups of the multivalent polymer blocks react in reactions that are orthogonal relative to the reactive groups present in the tumor cell-attractant and the cancer therapeutic agent.

In some embodiments, the precursor compound includes a dendritic PEG, a star PEG, or a comb PEG. Multiple examples of such structures are known in the art. A “dendritic poly(ethylene glycol),” also referred to herein as “dendritic PEG”, refers to a highly branched multi-arm poly(ethylene glycol) having a tree-like structure. Multiple examples of such structures are known in the art. A “comb poly(ethylene glycol),” also referred to herein as “comb PEG,” refers to a multi-arm poly(ethylene glycol) having a main chain with multiple trifunctional branch points from each of which a linear arm emanates. The term “star poly(ethylene glycol)”, also referred to herein as “star PEG”, refers to a multi-arm poly(ethylene glycol) having a central branch point, which may be a single atom or a chemical group, from which linear arms emanate.

In some embodiments, the hydrogel matrix can be assembled by crosslinking one or more precursor compounds, such as, for example, a multi-arm PEG, for example, a multi-arm star PEGs synthesized by ethoxylation of tripentaerythritol (8ARM(TP) PEG), hexaglycerol (8ARM PEG), dipentaerythritol (6ARM PEG), pentaerythritol (4ARM PEG), or glycerol (3ARM PEG). The multi-arm PEG precursor compounds can be functionalized with multiple reactive groups and can have, for example, four, six, or eight arms and a molecular weight of from about 5,000 to about 25,000. Compounds suitable for use as multi-arm PEG precursors are known in the art.

In some embodiments, PEG is used in the hydrogel matrix of the present disclosure because it can exhibit minimal or no intrinsic biological activity due to the non-adhesive and non-immunogenic nature of the polymer chains. The polymers forming the hydrogel can be linear or branched, and can be modified at some or all the hydroxyl termini with a variety of functional groups to alter their biological activity and chemical/enzymatic reactivity. In some embodiments, large biomolecules such as antibodies, growth factors, integrin binding sites, and degradation sites can be introduced into the backbone, pendant from sidechains to the backbone, and/or on the terminus of the polymers forming the hydrogel to bestow a tailored biological effect.

The hydrogel matrix of the present disclosure can be polymerized, for example, using bio-orthogonal reactions, which refer to reactions whose components have no endogenous representation in biological molecules, cells, or organs. One such bio-orthogonal chemistry is Strain Promoted Azide Alkyne Cyclo Addition (SPAAC) click chemistry. This type of reaction allows for bio-orthogonal hydrogel polymerization under aqueous conditions at neutral pH without the use of a catalyst. This reaction takes advantage of the spontaneous covalent bonding of azide groups to molecules possessing strained rings, such as cyclo-octyne and bi-cyclononyne (BCN). By functionalizing the ends of a polymer chain with BCN for example, azide-functionalized molecules can covalently bond with the ends of the polymer chain itself. As azides are not naturally found in the human body, this click reaction can take place in the presence of tissue and cells with no risk of toxic side reactions and by-products.

In some embodiments, the multivalent polymer building block and the crosslinker are reacted together via a reaction such as azide-alkyne cycloaddition, oxime ligation, hydrazide formation, thiol-maleimide, michael-type addition, thiol-ene, thiol-yne, strain-promoted alkyne-nitrone cycloaddition (SPANC), strain-promoted, azide-alkyne cycloaddition (SPAAC), copper(I)-catalyzed azide-alkyne cycloaddition (CuAAC), staudinger ligation, tetrazine-cyclooctene, diels-alder, inverse electron-demand diels-alder, native chemical ligation, cinnamate/coumarin/anthracine dimerization, amide formation through amine reacting with activated ester (e.g., N-hydroxysuccinimidyl esther, NHS), and enzymatic crosslinking (e.g., sortase ligation). The reaction of the multivalent polymer building block and the crosslinker can be orthogonal to the reaction of the therapeutic agent and/or the cancer-cell attractant to the hydrogel matrix. The multivalent polymer building block and the crosslinker can have complementary reactive groups, and can react with one another under appropriate reaction conditions, which are readily understood by a person of ordinary skill in the art.

In some embodiments, the multivalent polymer building block is a compound of Formula (I):

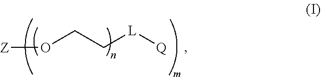

wherein:

Q, at each occurrence, is a reactive group;

n, at each occurrence, is independently an integer of from 1 to 50;

m, at each occurrence, is independently an integer of from 2 to 20,

Z is multi-arm core; and

L, at each occurrence, is independently absent or a linker group comprising 2-100 backbone atoms selected from C, N, O, S, and P.

In some embodiments, Q, at each occurrence, is independently a reactive group independently selected from SH, OH, amino, COOH, ester (e.g., an activated ester), N₃, optionally substituted maleimide, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, Q, at each occurrence, is independently a reactive group selected from SH, OH, amino, COOH, N₃, optionally substituted heteroaryl (e.g., tetrazine), optionally substituted C₃-C₆ alkenyl, optionally substituted C₂-C₆ alkynyl, and/or optionally substituted C₈-C₁₂ cycloalkynyl reactive group. In some embodiments, Q, at each occurrence, is independently a reactive group selected from SH, OH, amino, COOH, N₃, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, Q, at each occurrence, is independently a reactive group selected from SH, OH, amino, N₃, optionally substituted heteroaryl (e.g., tetrazine), ethynyl, optionally substituted C₃-C₆ alkynyl, and/or optionally substituted C₈-C₁₂ cycloalkynyl reactive group. In some embodiments, Q, at each occurrence, is independently a reactive group selected from N₃ and optionally substituted C₈-C₁₂ cycloalkynyl reactive group.

In some embodiments, n in Formula (I), at each occurrence, is independently an integer of from 1 (e.g., from 3, from 5, from 10, from 20, from 30, or from 40) to 50 (e.g., to 40, to 30, to 20, to 10, to 5, or to 3).

In some embodiments, m in Formula (I), is an integer of from 2 (e.g., from 3, from 4, from 5, from 6, from 7, from 8, from 9, from 10, from 12, from 14, from 16, or from 18) to 20 (e.g., to 18, to 16, to 14, to 12, to 10, to 9, to 8, to 7, to 6, to 5, to 4, or to 3).

In some embodiments, L in formula (I), at each occurrence, is independently absent or a linker group comprising 2 to 75 (e.g., 2 to 50, 2 to 25, 2 to 10) backbone atoms selected from C, N, O, S, and P (e.g., C, N, and O).

In some embodiments, L in Formula (I) can include one or more cleavable groups, such as an ester, an amide, a disulfide, an acetal, a ketal, an oxime, or a hydrazone group. The cleavable group can be a hydrolytically cleavable group. In some embodiments, the cleavable group is cleavable upon contact with an external agent.

In some embodiments, Z in formula (I) is a sugar alcohol. In some embodiments, Z in formula (I) is tripentaerythritol, hexaglycerol, pentaerythritol, or glycerol. In certain embodiments, Z in formula (I) is C(CH₂)₄ or an optionally substituted C₂-C₁₀ alkylene (e.g., a C₂ alkylene, a C₄ alkylene, a C₆ alkylene, a C₈ alkylene, or a C₁₀ alkylene). In certain embodiments, Z in formula (I) is C(CH₂)₄ or an optionally substituted C₂-C₆ alkylene.

In some embodiments, the multivalent polymer building block is a compound of Formula (IA):

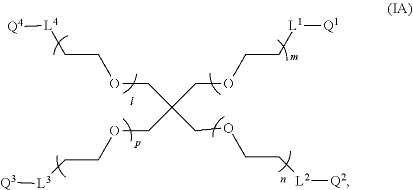

wherein:

Q¹, Q², Q³, and Q⁴ are each a reactive group independently selected from SH, OH, amino, COOH, ester (e.g., an activated ester), N₃, optionally substituted maleimide, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group;

l, m, n, and p are each independently integers of from 1 to 50; and

L¹, L², L³, and L⁴ are each independently linker groups including 2-100 backbone atoms independently selected from C, N, O, S, and P.

In some embodiments, Q¹, Q², Q³, and Q⁴ are each independently a reactive group selected from SH, OH, amino, COOH, ester (e.g., an activated ester), N₃, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, Q¹, Q², Q³, and Q⁴ are each independently a reactive group selected from SH, OH, amino, COOH, N₃, optionally substituted heteroaryl (e.g., tetrazine), optionally substituted C₃-C₆ alkenyl, optionally substituted C₂-C₆ alkynyl, and/or optionally substituted C₈-C₁₂ cycloalkynyl reactive group. In some embodiments, Q¹, Q², Q³, and Q⁴ are each independently a reactive group selected from SH, OH, amino, COOH, N₃, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, Q¹, Q², Q³, and Q⁴ are each independently a reactive group selected from SH, OH, amino, N₃, optionally substituted heteroaryl (e.g., tetrazine), ethynyl, optionally substituted C₃-C₆ alkynyl, and/or optionally substituted C₈-C₁₂ cycloalkynyl reactive group. In some embodiments, Q¹, Q², Q³, and Q⁴ are each independently a reactive group selected from N₃ and optionally substituted C₈-C₁₂ cycloalkynyl reactive group.

In some embodiments, l, m, n, p in Formula (IA) is each independently an integer of from 1 (e.g., from 3, from 5, from 10, from 20, from 30, or from 40) to 50 (e.g., to 40, to 30, to 20, to 10, to 5, or to 3).

In some embodiments, L¹, L², L³, and L⁴ in formula (IA) is each independently absent or a linker group comprising 2 to 75 (e.g., 2 to 50, 2 to 25, 2 to 10) backbone atoms selected from C, N, O, S, and P (e.g., C, N, and O).

In certain embodiments, L¹-Q¹, L²-Q², L³-Q³, and L⁴-Q⁴ are each independently represented by formulae:

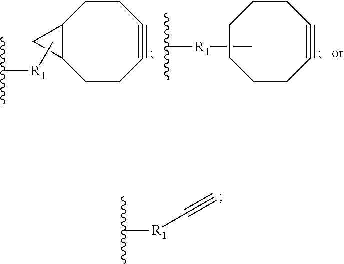

wherein R¹ is a linker group including 2-90 (e.g., 2 to 50, 2 to 25, 2 to 10) backbone atoms independently selected from C, N, O, S, and P (e.g., C, N, and O).

In some embodiments, one or more of L, L¹, L², L³, and L⁴ in Formula (IA) includes one or more cleavable groups, such as an ester, an amide, a disulfide, an acetal, a ketal, an oxime, or a hydrazone group. The cleavable group can be a hydrolytically cleavable group. In some embodiments, the cleavable group is cleavable upon contact with an external agent.

In some embodiments, the multivalent polymer building block is a compound of Formula (IB):

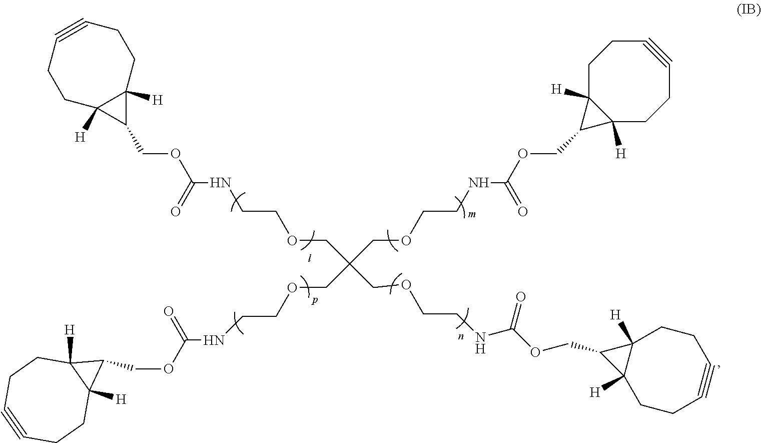

or an isomer or a tautomer thereof, wherein:

l, m, n, and p are each independently integers of from 1 to 50.

In some embodiments, l, m, n, p in Formula (IB) is each independently an integer of from 1 (e.g., from 3, from 5, from 10, from 20, from 30, or from 40) to 50 (e.g., to 40, to 30, to 20, to 10, to 5, or to 3).

In some embodiments, the hydrogel precursor is PEG-tetra-BCN. In some embodiments, the hydrogels disclosed herein are synthesized by contacting PEG-tetra-BCN with a crosslinking agent which comprises two azido groups and an azido derivative of one or more therapeutic agents, for example, under the conditions disclosed in U.S. Pat. No. 8,703,904, the disclosure of which is incorporated herein by reference.

As described above, the multivalent polymer building block can be reacted with a crosslinker to form the hydrogel matrix. The reactive groups on the crosslinker can be complementary to (i.e., can react to form covalent bonds with) the reactive groups on the multivalent polymer building blocks. For example, the crosslinker can have two or more reactive groups independently selected from SH, OH, amino, COOH, ester (e.g., an activated ester), N₃, optionally substituted maleimide, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group, so long that they are complementary to the reactive groups on the multivalent polymer building blocks.

In some embodiments, the crosslinker can be a compound of Formula (II)

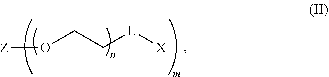

wherein:

X, at each occurrence, is a reactive group (complementary to the reactive groups on the multivalent polymer building blocks);

n, at each occurrence, is independently an integer of from 1 to 50;

m, at each occurrence, is independently an integer of from 2 to 20,

Z is a multi-arm core; and

L, at each occurrence, is independently absent or a linker group comprising 2-100 backbone atoms selected from C, N, O, S, and P.

In some embodiments, X, at each occurrence, is independently a reactive group (complementary to the reactive groups on the multivalent polymer building blocks) selected from SH, OH, amino, COOH, ester (e.g., an activated ester), N₃, optionally substituted maleimide, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, X, at each occurrence, is independently a reactive group (complementary to the reactive groups on the multivalent polymer building blocks) selected from SH, OH, amino, COOH, N₃, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, X, at each occurrence, is independently a reactive group (complementary to the reactive groups on the multivalent polymer building blocks) selected from SH, OH, amino, N₃, optionally substituted heteroaryl (e.g., tetrazine), ethynyl, optionally substituted C₃-C₆ alkynyl, and/or optionally substituted C₈-C₁₂ cycloalkynyl reactive group. In some embodiments, X, at each occurrence, is independently a reactive group (complementary to the reactive groups on the multivalent polymer building blocks) selected from N₃ and optionally substituted C₈-C₁₂ cycloalkynyl reactive group.

In some embodiments, n in Formula (II), at each occurrence, is independently an integer of from 1 (e.g., from 3, from 5, from 10, from 20, from 30, or from 40) to 50 (e.g., to 40, to 30, to 20, to 10, to 5, or to 3).

In some embodiments, m in Formula (II), is an integer of from 2 (e.g., from 3, from 4, from 5, from 6, from 7, from 8, from 9, from 10, from 12, from 14, from 16, or from 18) to 20 (e.g., to 18, to 16, to 14, to 12, to 10, to 9, to 8, to 7, to 6, to 5, to 4, or to 3).

In some embodiments, L in Formula (II), at each occurrence, is independently absent or a linker group comprising 2 to 75 (e.g., 2 to 50, 2 to 25, 2 to 10) backbone atoms selected from C, N, O, S, and P (e.g., C, N, and O).

In some embodiments, L in Formula (II) can include one or more cleavable groups, such as an ester, an amide, a disulfide, an acetal, a ketal, an oxime, or a hydrazone group. The cleavable group can be a hydrolytically cleavable group. In some embodiments, the cleavable group is cleavable upon contact with an external agent.

In some embodiments, Z in Formula (II) is a sugar alcohol. In some embodiments, Z in Formula (II) is tripentaerythritol, hexaglycerol, pentaerythritol, or glycerol. In certain embodiments, Z in Formula (II) is C(CH₂)₄ or an optionally substituted C₂-C₁₀ alkylene (e.g., a C₂ alkylene, a C₄ alkylene, a C₆ alkylene, a C₈ alkylene, or a C₁₀ alkylene). In certain embodiments, Z in (Formula (II) is C(CH₂)₄ or an optionally substituted C₂-C₆ alkylene.

In some embodiments, the crosslinker is a compound of Formula (IIA):

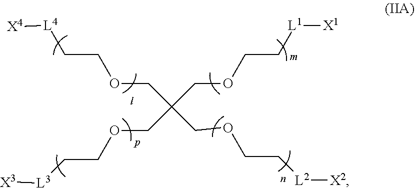

wherein:

X¹, X², X³, and X⁴ are each independently a reactive group from a group consisting of SH, OH, amino, COOH, ester (e.g., an activated ester), N₃, optionally substituted maleimide, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group;

l, m, n, and p are each independently an integer of from 1 to 50; and

L¹, L², L³, and L⁴ are independently a linker group comprising 2-100 backbone atoms selected from C, N, O, S, and P.

In some embodiments, X¹, X², X³, and X⁴ are each independently a reactive group (complementary to the reactive groups on the multivalent polymer building blocks) selected from SH, OH, amino, COOH, ester (e.g., an activated ester), N₃, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive group. In some embodiments, X¹, X², X³, and X⁴ are each independently a reactive group (complementary to the reactive groups on the multivalent polymer building blocks) selected from SH, OH, amino, COOH, N₃, optionally substituted heteroaryl (e.g., tetrazine), optionally substituted C₃-C₆ alkenyl, optionally substituted C₂-C₆ alkynyl, and/or optionally substituted C₈-C₁₂ cycloalkynyl reactive groups. In some embodiments, X¹, X², X³, and X⁴ are each independently a reactive group selected from SH, OH, amino, COOH, N₃, optionally substituted heteroaryl (e.g., tetrazine), optionally substituted C₃-C₆ alkenyl, ethynyl, optionally substituted C₃-C₆ alkynyl, and/or optionally substituted C₈-C₁₂ cycloalkynyl reactive groups. In some embodiments, X¹, X², X³, and X⁴ are each independently a reactive group selected from SH, OH, amino, N₃, optionally substituted heteroaryl (e.g., tetrazine), ethynyl, optionally substituted C₃-C₆ alkynyl, and/or optionally substituted C₈-C₁₂ cycloalkynyl reactive groups. In some embodiments, X¹, X², X³, and X⁴ are each independently a reactive group selected from N₃ and optionally substituted C₈-C₁₂ cycloalkynyl reactive groups.

In some embodiments, l, m, n, p in Formula (IIA) is each independently an integer of from 1 (e.g., from 3, from 5, from 10, from 20, from 30, or from 40) to 50 (e.g., to 40, to 30, to 20, to 10, to 5, or to 3).

In some embodiments, L¹, L², L³, and L⁴ in formula (IIA) is each independently absent or a linker group comprising 2 to 75 (e.g., 2 to 50, 2 to 25, 2 to 10) backbone atoms selected from C, N, O, S, and P (e.g., C, N, and O).

In some embodiments, one or more of L, L¹, L², L³, and L⁴ in Formula (IIA) includes one or more cleavable groups, such as an ester, an amide, a disulfide, an acetal, a ketal, an oxime, or a hydrazone group. The cleavable group can be a hydrolytically cleavable group. In some embodiments, the cleavable group is cleavable upon contact with an external agent.

In some embodiments, the crosslinker is a compound of Formula (IIB):

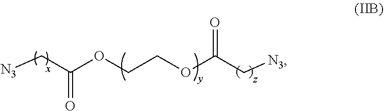

wherein:

x and z are each independently an integer of from 1 to 6, and

y is an integer of from 1 to 50.

In some embodiments, x and z are each independently 2, 3, 4, 5, or 6 (e.g., 2, 3, or 4; or 4)

In some embodiments, y in Formula (IIB) is an integer of from 1 (e.g., from 3, from 5, from 10, from 20, from 30, or from 40) to 50 (e.g., to 40, to 30, to 20, to 10, to 5, or to 3).

In some embodiments, the crosslinker is a compound of Formula (IIC)

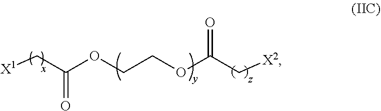

wherein:

X¹ and X² are each a reactive group independently selected from:

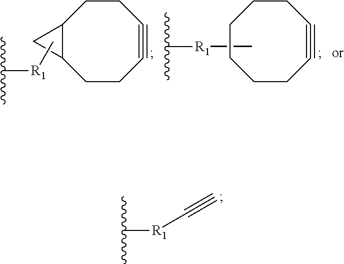

R¹ is absent or a linker group comprising 2-10 backbone atoms selected from C, N, O, S, and P;

x and z are each independently an integer of from 0 to 6; and

y is an integer of from 1 to 50.

In some embodiments, x and z in Formula (IIC) are each independently 1, 2, 3, 4, 5, or 6 (e.g., 2, 3, or 4; or 4).

In some embodiments, y in Formula (IIC) is an integer of from 1 (e.g., from 3, from 5, from 10, from 20, from 30, or from 40) to 50 (e.g., to 40, to 30, to 20, to 10, to 5, or to 3).

In some embodiments, R¹ in Formula (IIC) is absent.

In some embodiments, R¹ in Formula (IIC) is a linker group including from 2 to 10 (e.g., 2, 4, 6, 8, or 10; 2 to 8, 2 to 6, 2 to 4) backbone atoms selected from C, N, O, S, and P (e.g., C, N, or O).

In some embodiments, when the multivalent polymer building block and the crosslinker are reacted together to provide the hydrogel matrix, the hydrogel matrix includes a crosslinking moiety of Formula (III):

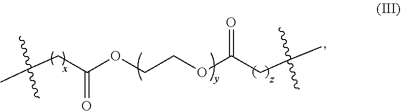

wherein x and z are each independently an integer selected from 1, 2, 3, 4, 5, and 6, and y is an integer of from 1 to 50.

In some embodiments, x and z in Formula (III) are each independently 2, 3, 4, 5, or 6 (e.g., 2, 3, or 4; or 4).

In some embodiments, y in Formula (III) is an integer of from 1 (e.g., from 3, from 5, from 10, from 20, from 30, or from 40) to 50 (e.g., to 40, to 30, to 20, to 10, to 5, or to 3).

In some embodiments, the optionally substituted C₈-C₂₀ cycloalkyne in the multivalent polymer building block and/or the crosslinker is each independently derived from a compound have a structure selected from:

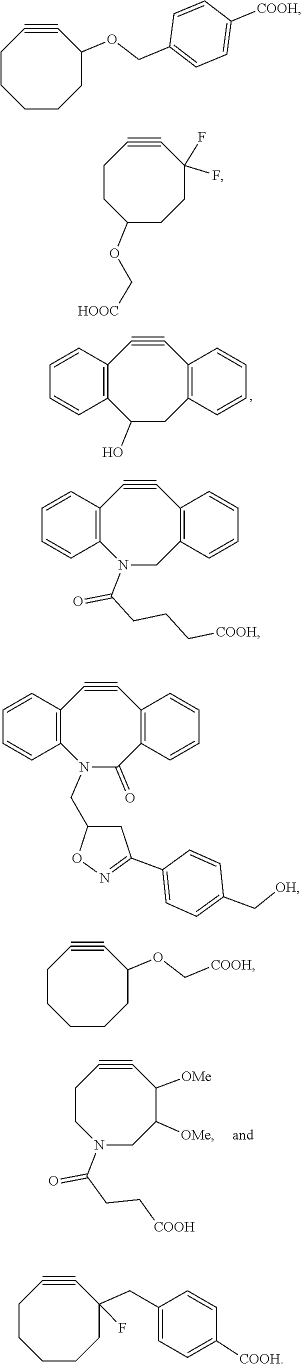

In some embodiments, when a hydrogel is formed from a multivalent polymer building block of a compound of Formula (IB) and a crosslinker of a compound of Formula (IIB), the hydrogel matrix structure can be formed when about 60% or more of the bicyclononynyl (BCN) groups are covalently bound to the crosslinker. The remaining BNC groups can be reacted with a therapeutic agent, a cancer cell attractant, or both.

In some embodiments, the hydrogel matrix includes a cleavable linkage, such as an ester, an amide, a disulfide, an acetal, a ketal, an oxime, or a hydrazone linkage. In certain embodiments, the hydrogel matrix includes ester linkages. In some embodiments, the cleavable linkage can be cleaved by hydrolysis, or a catalytic process (e.g., an enzyme-mediated hydrolysis and/or oxidation).

Therapeutic Agent and Cancer Cell-Attractant

As discussed above, a therapeutic agent can be associated to the hydrogel matrix of the immunotherapy delivery hydrogel system. In some embodiments, a tumor cell-attractant can be conjugated to the hydrogel matrix. Any suitable therapeutic agent can be associated with and any tumor cell-attractant can be conjugated to the hydrogel matrix to provide immunotherapy delivery hydrogel system. For example, the therapeutic agent and/or the tumor cell-attractant can include a protein and/or a peptide, which can range from very small to very large. For example, small peptides such as cysteine-dense knotted peptides (knottins) and small molecule inhibitors to a tumor-specific target can be used. Larger therapeutic molecules include cytokines, e.g., IFNg which could turn a “cold” tumor into a “hot” tumor by forcing expression of MHC I. In some embodiments, the therapeutic agent is IL-4, release of which from the hydrogel matrix can shift the local immune cells into an anti-tumor phenotype. In some embodiments, the agents include antibodies or a nanobodies that specifically neutralize and opsonize tumor cells.

Therapeutic Agent

In some embodiments, the therapeutic agent includes an antibody or a binding fragment thereof, an immune stimulatory molecule; a bispecific T-cell engager, an immune checkpoint molecule, an immune cell (e.g., modified T-cells and/or NK cells), or any combination thereof.

In some embodiments, the therapeutic agent includes an immune checkpoint molecule (e.g., an immune checkpoint inhibitor). As used herein, an “immune checkpoint inhibitor” is an agent that can block certain proteins expressed by cancer cells which prevent immune cells from killing cancer cells, for example, by phagocytic clearance by macrophages. In some embodiments, the one or more immune checkpoint inhibitors is an agent that blocks CD47. CD47 is a cell-surface protein that serves as a “do not eat me” signal when engaged by its ligand, SIRPα, on phagocytic macrophages.

In some embodiments, any agent that disrupts the CD47/SIRPα axis and/or decreases or prevents macrophage phagocytosis and renders tumor cells more sensitive to innate immune surveillance can be used as an immune checkpoint inhibitor in the immunotherapy delivery hydrogel system. In some instances, CD47 is the dominant macrophage checkpoint overexpressed on certain cancer cells. In some embodiments, the one or more immune checkpoint inhibitors is an anti-CD47 antibody or a binding fragment thereof, an anti-CD47 aptamer, or a combination thereof. In some instances, the one or more immune checkpoint inhibitors is a monoclonal anti-CD47 antibody or a binding fragment thereof. For example, exemplary immune checkpoint inhibitors include anti-CD47 monoclonal antibody (mAb), anti-SIRPα mAb, and SIRPα-Fc fusion protein, examples of each of which are known in the art.

In some embodiments, the antibody or a binding fragment thereof can include an immune checkpoint inhibitor, an anti-CD47 antibody, an anti-CD24 antibody, an anti-PD-L1 antibody, and/or an anti-B7H3 antibody, or a binding fragment thereof. The anti-CD47 antibody or a binding fragment thereof includes a sequence having at least 90% homology to SEQ ID NO: 1. The anti-CD47 antibody or a binding fragment thereof can include an enzyme-recognizable sequence (e.g., a sortase recognition sequence) at the C-terminus. In some embodiments, the anti-CD47 antibody molecule includes a C-terminus linker that includes a cleavable group, wherein the cleavable group is a group cleavable under biological conditions. The C-terminus linker can further include a reactive group, such as an azido group or an alkyne.

In some embodiments, the immune checkpoint inhibitor includes an agent that blocks intracellular signaling domains of CD47's cognate receptor, SIRPα, and/or other ITIM-containing receptors. The ITIM can include a phosphatase, Shp-1 which deactivates the positive signal from the TCR, FcR, and the like. Thus, in some embodiments, the immune checkpoint inhibitors include inhibitors of Shp-1, for example, sodium stibogluconate (Pentostam), NSC87877720, and TPI-1. In some embodiments, the immune checkpoint inhibitor is an agent that selectively inhibits Shp-1 and does not inhibit Shp-2.

In some embodiments, the immune checkpoint inhibitor inhibits one or more hematopoietic-specific Src family kinases (SFK) which phosphorylate the ITIM domain and/or Shp-1. Suitable SFK kinases targeted by immune checkpoints inhibitors include Fgr, Lyn, Hck, Blk, and Lck (Front Biosci. 2008; 13: 4426-4450, the disclosure of which is incorporated herein by reference). A number of SFK inhibitors is known in the art.

In some embodiments, the hydrogel compositions of the disclosure can comprise a masking molecule, for example, an immune checkpoint molecule, e.g., on the hydrogel's surface. In the event that a subject's body develops antibodies to the PEG backbone of the hydrogel, the hydrogel's presence can cause an unintended immune response that can result in the patient's own immune cells attacking the hydrogel. In some embodiments, the immune checkpoint molecules include PD-L1, CTLA-4, CD47, CD24, CD155, CD112, β2 Microglobulin (B2M), or any combination thereof.

In some embodiments, the immune checkpoint inhibitor includes a dual checkpoint inhibitor, i.e., an agent that acts by both downregulating CD47 on cancer cells and SIRP-α on monocytes/macrophages. A non-limiting example of such dual checkpoint inhibitor is RRx-001 or 2-bromo-1-(3,3-dinitroazetidin-1-yl)ethanone, disclosed in Cabrales P. RRx-001 Acts as a Dual Small Molecule Checkpoint Inhibitor by Downregulating CD47 on Cancer Cells and SIRP-α on Monocytes/Macrophages. Transl. Oncol. 2019; 12(4):626-632, the disclosure of which is incorporated herein by reference in its entirety.

In some embodiments, the therapeutic agent is an anti-CD47 antibody or a binding fragment thereof comprising a sequence having at least 90% homology to METDTLLLWVLLLWVPGSTGQVQLQESGPGLVKPSGTLSLTCAVSGVSIRSINWW NWVRQPPGKGLEWIGEIYHSGSTNYNPSLKSRVTISVDKSKNQFSLKLNSVTAAD TAVYYCARDGGIAVTDYYYYGLDVWGQGTTVTVSSASTKGPSVFPLAPSSKSTS GGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQYNSTYR VVSVLTVLHQDWLNGKEYKCKVSNKALPAPIEKTISKAKGQPREPQVYTLPPSRD ELTKNQVSLTCLVKGFYPSDIAVEWESNGQPENNYKTTPPVLDSDGSFFLYSKLTV DKSRWQQGNVFSCSVMHEALHNHYTQKSLSLSPGK (SEQ ID NO: 1) or METDTLLLWVLLLWVPGSTGQVQLQESGPGLVKPSGTLSLTCAVSGVSIRSINWW NWVRQPPGKGLEWIGEIYHSGSTNYNPSLKSRVTISVDKSKNQFSLKLNSVTAAD TAVYYCARDGGIAVTDYYYYGLDVWGQGTTVTVSSASTKGPSVFPLAPSSKSTS GGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPK DTLMISRTPEVTCVVVDVSHEDPEVKFNWYVDGVEVHNAKTKPREEQYNSTYR VVSVLTVLHQDWLNGKEYKCKVSNKALPAPIEKTISKAKGQPREPQVYTLPPSRD ELTKNQVSLTCLVKGFYPSDIAVEWESNGQPENNYKTTPPVLDSDGSFFLYSKLTV DKSRWQQGNVFSCSVMHEALHNHYTQKSLSLSPGKGGLPETGGHHHHHH (SEQ ID NO: 2). In some embodiments, the anti-CD47 antibody or a binding fragment thereof is an antibody disclosed in U.S. Pat. No. 9,650,441, the disclosure of which is incorporated herein by reference in its entirety. In some embodiments, the anti-CD47 antibody or a binding fragment thereof comprises a sortase recognition sequence at the C-terminus, for example, LPXTG, wherein X is any amino acid (SEQ ID NO: 3).

In some embodiments, the therapeutic agent is a bispecific agent, such as Bispecific T cell engager (BiTE). In some embodiments, the BiTEs is Blinatumomab or Solitomab. Universally, BiTEs are cleared from the body rapidly and require constant infusion via a drug pump. Sustained release from the hydrogel composition including such BiTEs within the tissue provides a more convenient method of administration.

In some embodiments, the hydrogel compositions can release an immune stimulatory molecule that can educate the immune cells, such as donor immune cells entrapped in the hydrogel, to target cancer antigens so that when the immune cells migrate to lymph nodes, they can eliminate metastatic cancer cells. In some embodiments, the immune stimulatory molecules are selected from INFg, IL-2, IL-4, IL-15, a fusion of IL-15_IL-15 Receptor Alpha, and combinations thereof. The immune cell (e.g., lymphocyte) can be entrapped in the hydrogel matrix. The stimulatory molecule can stimulate unstimulated immune cells. In some embodiments, the immune stimulatory molecule can convert immune cells present within the local hydrogel microenvironment into an anti-cancer phenotype.

In some embodiments, the hydrogel system includes cells entrapped or encapsulated within the hydrogel matrix. For example, the chemistry, such as SPAAC disclosed herein, involved in polymerizing the hydrogel matrix can be non-toxic and cytocompatible. Cells can be present in the reaction mixture, and the hydrogel can be formed, e.g., polymerized, around them. For example, immune cells within the gel can be included to educate the patient's immune system to the tumor cells residing in the surrounding microenvironment. The cells could be unstimulated immune cells from a donor, such as NK Cells, dendritic cells, T Cells, and macrophages, which together would comprise a full immune response (innate and adaptive). In some embodiments, the cells can be pre-engineered to specifically home in on tumor cells, such as CAR T-Cells.

Cancer Cell Attractant

In some embodiments, the cancer cell attractant includes a chemokine, a cytokine, an anti-cancer therapeutic agent, an immune stimulatory agent, or a combination thereof. In some embodiments, the cancer cell attractant includes a chemokine. The chemokine can include, for example, mCXCL12, CXCL12, CCL2, mCCL2, CX3CL1, mCX3CL1, or any combination thereof. The chemokine can include an enzyme recognition sequence (e.g., a sortase recognition sequence) at the C-terminus. In some embodiments, the chemokine comprises a sortase recognition sequence at the C-terminus, for example, a LPXTG sequence (SEQ ID NO: 3), wherein X is any amino acid. In some embodiments, the chemokine includes a C-terminus linker that includes a cleavable group, wherein the cleavable group is a group cleavable under biological conditions. The C-terminus linker can further include a reactive group, such as an azido group or an alkyne.

In some embodiments, the tumor cell-attractant, the cancer therapeutic agent, or both, are each released from the hydrogel matrix at a predetermined rate. The predetermined rate can be tuned depending on the length of a crosslinker's carbon chain (e.g., longer carbon chains lead to slower release rate), the amount of crosslinker relative to the multivalent polymer building block; the x and/or z integer values in the crosslinker of Formula (IIB) and/or (IIC) (where the higher the integer value, the slower the cleavage); the amount of the tumor cell-attractant, the cancer therapeutic agent, or both, relative to the polymer matrix; and/or the type of cleavable linkage. In some embodiments, when the tumor cell-attractant, the cancer therapeutic agent, or both, are not covalently bound to the hydrogel matrix, an increased amount of crosslinker can provide a slower release of the tumor cell-attractant, the cancer therapeutic agent, or both. In some embodiments, it is the structure of the cleavable crosslinker that is varied to tune the degradation half-lives (e.g., from hours to months) of the immunotherapy delivery hydrogel system or the hydrogel matrix.

The tumor cell-attractant, the cancer therapeutic agent, or both, can be covalently bound to the hydrogel matrix. In some embodiment, the tumor cell-attractant, the cancer therapeutic agent, or both, are hydrolytically or enzymatically cleavable from the hydrogel matrix. In some embodiments, the immunotherapy delivery hydrogel system has more than one kind of cleavable linkage. In some embodiments, the tumor cell-attractant, the cancer therapeutic agent, or both are linked to the hydrogel matrix via an ester, an amide, a disulfide, an acetal, a ketal, an oxime, and/or a hydrazone linkage. In some embodiments, an amide linkage is cleavable in the presence of a protease. In certain embodiments, an amide linkage provides a permanent linkage to a hydrogel matrix. Without wishing to be bound by theory, it is believed that the cleavage rate of the linkages themselves, from fastest to slowest, ranges from a hydrazone linkage, a disulfide linkage, an ester linkage, a ketal linkage, an acetal linkage, an oxime linkage, to an amide linkage.

In some embodiments, the tumor cell-attractant, the cancer therapeutic agent, or both, are physically entrapped in the hydrogel matrix. In certain embodiments, the tumor cell-attractant, the cancer therapeutic agent, or both, are associated with the hydrogel matrix via non-covalent interactions (e.g., electrostatic, π-, van der Waals, and/or hydrophobic interactions).

In certain embodiments, the hydrogel matrix can further include one or more near-IR fluorescent molecules linked to the hydrogel matrix.

In some embodiments, the immunotherapy delivery hydrogel system includes a hydrogel matrix; a chemokine conjugated to the hydrogel matrix; and a macrophage checkpoint antibody or a fragment thereof associated with the hydrogel matrix.

The mole ratio of the total therapeutic agent to total cancer cell attractant in a given immunotherapy delivery hydrogel system can be 3 or more (i.e., 3 or more moles of therapeutic agent for every mole of cancer cell attractant, 4 or more moles of therapeutic agent for every mole of cancer cell attractant, 5 or more moles of therapeutic agent for every mole of cancer cell attractant, or 6 or more moles of therapeutic agent for every mole of cancer cell attractant).

The total therapeutic agent can be present in the hydrogel matrix in an amount of 10 μM or more (e.g., 12 μM or more, 14 μM or more, 16 μM or more, 20 μM or more, 24 μM or more, 28 μM or more, or 32 μM or more).

The total cancer cell attractant can be present in the hydrogel matrix in an amount of 4 μM or more (e.g., 5 μM or more, 6 μM or more, 7 μM or more, or 8 μM or more).

In some embodiments, the mole ratio of the total therapeutic agent to total cancer cell attractant in a given immunotherapy delivery hydrogel system is 3 or less (i.e., 2 or less moles of therapeutic agent for every mole of cancer cell attractant, 1 or less moles of therapeutic agent for every mole of cancer cell attractant, or 0.5 or less moles of therapeutic agent for every mole of cancer cell attractant).

In some embodiments, the total therapeutic agent is present in the hydrogel matrix in an amount of 10 μM or less (e.g., 8 μM or less, 6 μM or less, 4 μM or less, or 2 μM or less).

In some embodiments, the total cancer cell attractant can be present in the hydrogel matrix in an amount of 4 μM or less (e.g., 3 μM or less, 2 μM or less, or 1 μM or less).

When administered to a subject, the rate of release of the therapeutic agent from the immunotherapy delivery hydrogel system can be equal or greater than the rate of degradation of the hydrogel matrix, in a biological environment. In some embodiments, the hydrogel matrix has hydrolysable groups that have a slower rate of hydrolysis than the rate of hydrolysis of the cleavable group linking the therapeutic agent and/or the cancer cell attractant to the hydrogel matrix.

Methods of Making the Immunotherapy Delivery Hydrogel System

The polymerization reaction can be carried out by reacting the multivalent polymer building block with a crosslinker, one or more therapeutic agents including a reactive group orthogonal to the reactive groups of the multivalent polymer building block and the crosslinker, and/or one or more cancer cell attractant including a reactive group orthogonal to the reactive groups of the multivalent polymer building block and the crosslinker.

In some embodiments, the hydrogel is formed in vivo. For example, the hydrogel is formed by depositing a reaction mixture that includes the multivalent polymer building block, the crosslinker, one or more therapeutic agents including a reactive group orthogonal to the reactive groups of the multivalent polymer building block and the crosslinker, and/or one or more cancer cell attractant into a biological tissue; and polymerizing the reaction mixture within the biological tissue.

Linking the therapeutic agent and/or the cancer cell attractant to hydrogel matrix can be done in any suitable manner. For example, an azidoacid can be used as a linker to tether the therapeutic agent and/or the cancer cell attractant to the hydrogel matrix, such as the following azido acid:

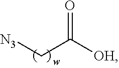

wherein w is an integer ranging from 1 to 10. In some embodiments, w is an integer ranging from 1 to 5. Such linkers can covalently attach to the reactive groups on the hydrogel matrix via the azide group and can form either esters or amides with the payload via the carboxylic acid, depending on the attachment chemistry used. The esters formed between the therapeutic agent and/or the cancer cell attractant and the azido acid linkers can hydrolyze at a rate determined by the length of the azido-acid carbon chain. In some embodiments, molecules conjugated to the linker as an amide are permanently linked to the gel.

In some embodiments, the linker (e.g., an azidoacid linker) can be attached to the C-terminus of a protein or polypeptide therapeutic agent and/or cancer cell attractant, using a sortase-mediated tagging (also referred to herein as “sortagging”) technique known in the art. A variety of soluble biomolecules with a C-terminal sortase recognition sequence, “LPXTG” can be prepared. The sortase enzyme recognizes this sequence and conjugates a triglycine (GGG) polypeptide to this tag. By synthesizing a GGG polypeptide with an additional amino acid containing a hydroxyl side chain (serine, threonine, tyrosine), esterification chemistry can be used to conjugate a linker, for example, an azidoacid to the hydroxyl group of the side chain.

In some embodiments, the peptide sequence of the therapeutic agent and/or the cancer cell-attractant includes the following structures, for example, incorporated at the C terminus:

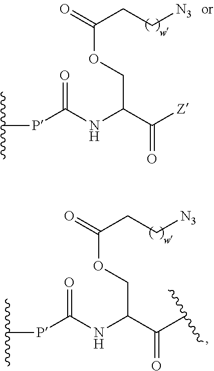

wherein P′ is absent or a peptide having from 1 to 10 amino acid residues,

Z′ is OH or NH₂, and

w′ is an integer ranging from 1 to 10.

In some embodiments, P′ can be a GGGR.

In some embodiments, P′ is absent.

In some embodiments, P′ is a peptide having from 1 to 8 amino acid residues (e.g., from 1 to 6 amino acid residues; from 1 to 4 amino acid residues, 3 amino acid residues, 4 amino acid residues, 5 amino acid residues, 6 amino acid residues, 7 amino acid residues, or 8 amino acid residues).

In some embodiments, Z′ is OH

In some embodiments, Z′ is NH₂.

In some embodiments, w′ is an integer ranging from 1 to 8 (e.g., from 1 to 6, from 1 to 4, from 1 to 3, 1, 2, 3, 4, 5, 6, 7, or 8).

In some embodiments, the linker group, i.e., a group linking the therapeutic agent with the hydrogel matrix has the structure represented by the formulae:

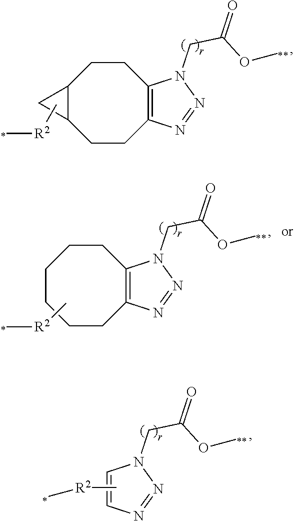

wherein r is 1, 2, 3, 4, or 5; and

R² is a linker group including 2-90 backbone atoms selected from C, N, O, S, and P;

* denotes the point of attachment to the hydrogel matrix; and

** denotes the point of attachment to the therapeutic protein agent, for example, the C-terminus or a side chain of an immune checkpoint inhibitor, such as anti-CD47.

In some embodiments, r is 1, 2, 3, or 4 (e.g., 1, 2, or 3; 1 or 2; or 2).

In some embodiments, R² is a linker group including 2 to 75 (e.g., 2 to 50, 2 to 25, 2 to 10) backbone atoms selected from C, N, O, S, and P (e.g., C, N, and O).

In some embodiments, R² includes PEG.

In some embodiments, sortagging provides a method to achieve site-specific conjugation of a linkers to a therapeutic agent and/or a cancer cell attractant. In some embodiments, other enzymatic and chemical methods can be used to couple these molecules together, either specifically or non-specifically, with varying degrees of coupling molecule-to-target protein ratios. For example, the following techniques used for the generation of Antibody Drug Conjugates (ADCs) can be employed as an alternative to sortase tagging. N-hydroxysuccinimide (NHS) chemistry can be used to permanently conjugate molecules non-specifically to lysines and the N-terminus of a protein of interest. Maleimide chemistry is a thiol-specific reaction that permanently couples molecules non-specifically to cysteines found on a particular protein. Carbohydrate-based conjugation can be utilized if the protein of interest is glycosylated. Its sugar residues can be modified chemically or enzymatically to generate a reactive “handle” where a molecule could be specifically conjugated. Non-canonical amino acids (NCAA) can be incorporated at a desired location in a recombinant protein of interest for site specific conjugation. One example is the NCAA, pAcF, which is a bioorthogonal electrophile that can specifically react with an aminoxy nucleophile to create an oxime linkage. Any other suitable enzymatic methods of coupling can be used, such as the use of Bacterial/Mammalian transglutaminase (BTGase/MTGase) or formylglycine generating enzyme (FGE). BTGase/MTGase can catalyze the addition of a molecule containing an alkyl primary amine to a glutamine side chain within a protein of interest. FGE recognizes a 6 residue “LCXPXR” amino acid tag and oxidizes the cysteine to make a reactive formylglycine, which is orthogonal for side-specific chemical conjugation. As used herein, an “orthogonal group” denotes a group that can form a covalent linkage, e.g., under biological conditions, with a specific type of a reactive group and not the other types of reactive groups. In some embodiments, the therapeutic agent can be attached using a method as disclosed in Shadish, J. A. & DeForest, C. A. Site-Selective Protein Modification: From Functionalized Proteins to Functional Biomaterials. Matter, 2, 50-77 (2020), the disclosure of which is incorporated herein by reference in its entirety.

In some embodiments, the therapeutic agent and/or a cancer cell attractant are reacted with the hydrogel matrix via a reaction such as azide-alkyne cycloaddition, oxime ligation, hydrazide formation, thiol-maleimide, michael-type addition, thiol-ene, thiol-yne, strain-promoted alkyne-nitrone cycloaddition (SPANC), strain-promoted, azide-alkyne cycloaddition (SPAAC), copper(I)-catalyzed azide-alkyne cycloaddition (CuAAC), staudinger ligation, tetrazine-cyclooctene, diels-alder, inverse electron-demand diels-alder, native chemical ligation, cinnamate/coumarin/anthracine dimerization, amide formation through amine reacting with activated ester (e.g., N-hydroxysuccinimidyl esther, NHS), and enzymatic crosslinking (e.g., sortase ligation). The reaction of the therapeutic agent and/or a cancer cell attractant with the hydrogel matrix can be orthogonal to the reaction of the multivalent polymer building blocks and the crosslinker.

The hydrogel systems and/or the hydrogel matrices of the disclosure can be assembled in any suitable manner. In some embodiments, the hydrogel matrix or the hydrogel system are assembled by crosslinking the multivalent polymer building blocks described herein. In some embodiments, the hydrogel systems and/or the hydrogel matrices described herein are formed from the multivalent polymer building blocks and crosslinker, which can occur without the use of an initiator, or optionally in combination with external initiation (e.g., with initiated multivalent polymer building blocks). In some embodiments, the hydrogels disclosed herein include gels that spontaneously form through non-covalent interactions and form physical crosslinks.

In some embodiments, the release of the therapeutic agent and/or the cancer cell attractant from the hydrogel does not affect the overall hydrogel architecture, i.e., the structure of the hydrogel matrix. The hydrogel matrix can remain permanently in place in the body at the site of hydrogel injection. In some embodiments, the hydrogel matrix can be cleaved, for example, hydrolytically, to be eliminated from the body after the release of the therapeutic agent and/or the cancer cell attractant. The rates of the hydrolysis of the hydrogel and the release of the therapeutic agent can be controlled, for example, by the length of a crosslinker's carbon chain (e.g., longer carbon chains lead to slower release rate), the amount of crosslinker relative to the multivalent polymer building block; the x and/or z integer values in the crosslinker of Formula (IIB) and/or (IIC) (where the higher the integer value, the slower the cleavage); the amount of the tumor cell-attractant, the cancer therapeutic agent, or both, relative to the polymer matrix; and/or the type of cleavable linkage. In some embodiments, when the tumor cell-attractant, the cancer therapeutic agent, or both, are not covalently bound to the hydrogel matrix, an increased amount of crosslinker can provide a slower release of the tumor cell-attractant, the cancer therapeutic agent, or both. In some embodiments, it is the structure of the cleavable crosslinker that can be varied to tune the degradation half-lives (e.g., from hours to months) of the immunotherapy delivery hydrogel system or the hydrogel matrix.

In some embodiments, the immunotherapy delivery hydrogel system of the disclosure includes functionalized polyethylene glycol (PEG), polyvinyl alcohol (PVA), polyethylene glycol-diacrylate (PEGDA), PEG methacrylate (PEGMA), poly(hydroxyethyl methacrylate) (pHEMA), polyethylene glycol methyl ether methacrylate (PEGMEM), poly(pentaerythritol triacrylate), poly(N-isopropylacryl amide) (PNIPAAm), or combinations thereof.

### Methods of Making Recombinant Proteins

In some embodiments, the present disclosure provides a method of making a recombinant protein. For example, an enzyme-recognizable chemokine and/or antibody can be made in mammalian cells. The amino acid sequence for the chemokine or a domain sequence for an antibody having a conjugated enzyme-recognizable label (e.g., via a hydrolysable linker) at the C-terminus can be obtained. Gene fragments containing the sequences with C-terminal enzyme recognition sites can be inserted into plasmids, and the plasmids can be purified and transduced into a suitable mammalian cell line for protein expression. Supernatant solutions containing the molecules of interest can be collected and purified. In some embodiments, the purified chemokine and/or antibody can then be reacted in the presence of an enzyme and an enzyme-recognizable reactant having a reactive group (e.g., an azide-containing enzyme-recognizable reactant) to provide a chemokine and/or antibody having a reactive group (e.g., an azide group), which can then be purified.

For example, sortaggable mCXCL12 and CD47mAbs can be made in mammalian cells as outlined below. The mature amino acid sequence for mCXCL12 can be obtained from NCBI, GeneID 20315. The heavy chain variable domain (VH) sequence for 2.3D11 CD47mAb can be obtained from U.S. Pat. No. 9,650,441, incorporated herein by reference in its entirety. Gblocks (IDT) containing these sequences with C-terminal sortase recognition sites can be inserted into double digested expression plasmids using Gibson assembly (NEB). Assembled plasmids can be transformed into chemically competent STBL3 E. coli (Thermo Fisher Scientific) and sequenced for accuracy. Plasmids can be subsequently maxiprepped (Qiagen, 12162) and transduced into HEK293T for protein expression. Supernatants containing the molecules of interest can be collected and purified on AKTA FPLC (Cytiva). Purified chemokines and antibodies can be buffer exchanged into sortase reaction buffer (e.g., 50 mM Tris, 125 mM NaCl, 10 mM CaCl2) pH 7.5). In some embodiments, 50 μM mCXCL12 or CD47mAb can be reacted with, for example, 2.5 μM Sortase 5M and 1 mM GGGRS-4-azidoester in 500 μL SrtA reaction buffer for 2 hr at 37 degrees Celsius in a shaking incubator. 500 μL of Ni-NTA beads can be added to the completed reaction, for example, for 1 hour at 4 degrees Celsius, to sequester any un-reacted antibody, chemokine and Sortase 5M. The reaction can be spun (e.g., at 10 k G's for 2 minutes) and the supernatant containing the linked chemokine/antibody conjugates can be removed and buffer exchanged (e.g., back into PBS) for downstream use. While a specific procedure is described herein, it is understood that the procedure can be generalized as known to a person of skill in the art.

In some embodiments, the enzyme-recognizable chemokine and/or antibody can be made in bacteria, such as E. coli. As an example, the sequence of a chemokine containing an enzyme-recognizable sequence at the C-terminal can be inserted into expression plasmids, which can then be inserted into a protein ligation construct (e.g., a STEPL construct) to express the chemokine as a fusion protein, in a second plasmid. The assembled plasmids can be transformed into a chemically competent bacterium. The plasmids can then be purified and transformed into another suitable bacterium, which can be grown in liquid culture and induced. The induced bacterium can be pelleted, lysed, and sonicated to release the final protein.

For example, the steps involved for making sortaggable mCXCL12 in E. coli are outlined below. The mature amino acid sequence for mCXCL12 was obtained from NCBI, GeneID 20315. Gblocks (IDT) containing these sequences with C-terminal sortase recognition sites and a 6×His-Tag can be inserted into double digested expression plasmids using Gibson assembly (NEB). In some embodiments, these sequences can be inserted into STEPL constructs where the chemokine is expressed as a fusion protein with sortase, by itself in a pET-29 plasmid. The assembled plasmids can be transformed into chemically competent STBL3 E. coli (Thermofisher) and sequenced for accuracy. Plasmids can be subsequently maxiprepped (Qiagen, 12162) and transformed into Shuffle T7 Express E. coli, grown in liquid culture to O.D 0.5-0.8 and induced with 0.4 mM IPTG overnight at 16 degrees Celsius. Induced E. coli can be pelleted, lysed, and sonicated to release solubilized protein. While chemokines are described herein, it is understood that antibodies can be made in an analogous manner.

Where a chemokine and/or antibody is expressed by itself, the chemokine and/or antibody can be purified, and reacted in the presence of an enzyme and an enzyme-recognizable reactant having a reactive group (e.g., an azide-containing enzyme-recognizable reactant) to provide a chemokine and/or antibody having a reactive group (e.g., an azide group), which can then be purified.

As an example, for chemokines expressed by themselves, Ni-NTA resin can be used to purify the chemokines (e.g., his-tagged chemokines) from contaminating bacterial protein, which can be washed, eluted and buffer exchanged into sortase reaction buffer (50 mM Tris, 125 mM NaCl, 10 mM CaCl2) pH 7.5). For example, 50 μM mCXCL12 can be reacted with 2.5 uM Sortase 5M and 1 mM GGGRS-4azidoester in 500 μL SrtA reaction buffer for 2 hr at 37 degrees Celsius in a shaking incubator. 500 μL of Ni-NTA beads can be added to the completed reaction (e.g., for 1 hour at 4 degrees Celsius) to sequester any un-reacted chemokine, reactant, and Sortase 5M. The reaction can be spun (e.g., at 10 k G's for 2 minutes) and the supernatant containing the linked chemokine/antibody conjugates can be removed and buffer exchanged (e.g., back into PBS) for downstream use.

Where a chemokine and/or antibody is expressed as a STEPL fusion protein, as an example, Ni-NTA resin can be used to purify fusion proteins from contaminating bacterial protein, washed and incubated (e.g., in 2 mL STEPL buffer (20 mM Tris, 125 mM NaCl, 100 μM CaCl₂)) containing 10 mM GGGGRS-4azidoester). As an example, the reaction can be shaken at 37 degrees for 4 hours. Conjugated mCXCL12-4-azidoester can be released from the resin via the STEPL reaction, collected and buffer exchanged (e.g., into PBS) for downstream use.

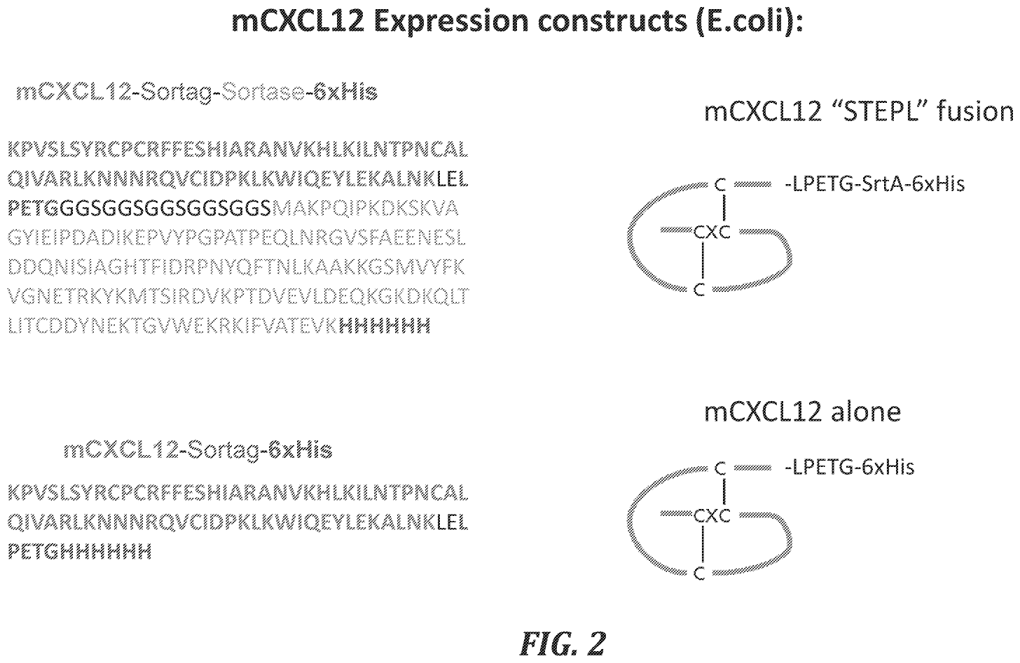

As shown in FIG. 2, an example of a mCXCL12 “STEPL” fusion protein sequence is KPVSLSYRCPCRFFESHIARANVKHLKILNTPNCALQIVARLKNNNRQVCIDPKLK WIQEYLEKALNKLELPETGGGSGGSGGSGGSGGSMAKPQIPKDKSKVAGYIEIPD ADIKEPVYPGPATPEQLNRGVSFAEENESLDDQNISIAGHTFIDRPNYQFTNLKAA KKGSMVYFKVGNETRKYKMTSIRDVKPTDVEVLDEQKGKDKQLTLITCDDYNE KTGVWEKRKIFVATEVKHHHHHH (SEQ ID NO. 9); and an example of a mCXCL12 sequence is

(SEQ ID NO. 10)
KPVSLSYRCPCRFFESHIARANVKHLKILNTPNCA
LQIVARLKNNNRQVCIDPKLKWIQEYLEKALNKLE
LPETGHHHHHH.

### Methods of Treatment

In another aspect, provided herein a method of treatment of a malignancy or an immune disorder, including administering a therapeutically effective amount of the immunotherapy delivery hydrogel system to a patient in need thereof. In some embodiments, the methods include contacting a biological tissue, such as a tumor or aa tumor recession cavity, with an immunotherapy delivery hydrogel system of the disclosure.

In some embodiments, the method first includes surgically resecting the tumor, then filling the resection cavity with the immunotherapy delivery hydrogel system disclosed herein. When administered, the immunotherapy delivery hydrogel system can inhibit, decrease, or eliminate incompletely resected tumor cells within and proximal to the tumor resection cavity in the subject.

In some embodiments, the cancer has an upregulation of PD-L1, CTLA-4, CD47, CD24, CD155, CD112, β₂ Microglobulin (B2M), or any combination thereof.

In some embodiments, during administration, the immunotherapy delivery hydrogel system is at a temperature lower than the body temperature of the subject during administration. The hydrogel can be in a flowable format when administered. In certain embodiments, the immunotherapy delivery hydrogel system is at a temperature higher than the body temperature of the subject during administration. The hydrogel can be in a solid format when administered. In some embodiments, rather than administering the immunotherapy delivery hydrogel system, a reaction mixture of a multivalent polymer building block, the crosslinker, an optional initiator, a therapeutic agent, and/or a cancer cell attractant can be administered to the subject, and the immunotherapy delivery hydrogel system can be generated in vivo.

In some embodiments, the immunotherapy delivery hydrogel system is administered to a tumor tissue. In some embodiments, administration of the immunotherapy delivery hydrogel system to the subject occurs after surgical removal of a tumor or a portion of the tumor. In some embodiments, administration of the immunotherapy delivery hydrogel system is to a tumor directly and/or in close proximity to a tumor. In some embodiments, the immunotherapy delivery hydrogel system is administered into a tissue directly abutting a cavity formed by surgical removal of the tumor. The immunotherapy delivery hydrogel system can be administered into the tissue to a depth that can be readily determined by a person of skill in the art, for example, to a depth of up to 5 cm (e.g., up to 4 cm, up to 3 cm, up to 2 cm, or up to 1 cm).

In some embodiments, the number of crosslinking moieties in the immunotherapy delivery hydrogel system degrades at a rate of from 40% (e.g., from 30% from 20%, or from 10%) to 3% (e.g., to 10%, to 20%, or to 30%) per week. The immunotherapy delivery hydrogel system can release at least one of the cancer cell attractant and the cancer therapeutic agent from the hydrogel matrix. In some embodiments, the immunotherapy delivery hydrogel system simultaneously releases the cancer cell attractant and the cancer therapeutic agent from the hydrogel matrix. In some embodiments, the immunotherapy delivery hydrogel system releases a cancer cell attractant and a cancer therapeutic agent, and the cancer therapeutic agent attracts macrophages and/or microglia.

The immunotherapy delivery hydrogel system disclosed herein can be formed by polymerization of a hydrogel precursor composition within a biological tissue. For example, a solution of the multivalent polymer building block, the crosslinker, an optional initiator, a therapeutic agent, and/or a cancer cell attractant can be introduced into a cavity formed after surgical removal of the tumor, and thus the hydrogel matrix can be formed in situ. In some embodiments, the solution of the multivalent polymer building block, the crosslinker, an optional initiator, a therapeutic agent, and/or a cancer cell attractant can be injected into a solid tumor, a tissue adjacent to a solid tumor, a body cavity, or a tissue containing tumor cells. In some embodiments, the immunotherapy delivery hydrogel system can be formed on the surface of a solid tumor by applying a composition including one or more multivalent polymer building block, the crosslinker, an optional initiator, a therapeutic agent, and/or a cancer cell attractant to the surface of the solid tumor, for example, by spraying.

In some embodiments, the hydrogel compositions of the disclosure include cells expressing one or more chemokines which can be released from the hydrogel and form a macrophage- and/or tumor cell-attracting gradient of chemokines. In some embodiments, the hydrogel matrices provide a 3D cell culture scaffold for such chemokine-expressing cells, for example, cells that have been genetically engineered to overexpress one or more chemokines. A number of such 3D biocompatible hydrogel scaffolds is known in the art, for example, the hydrogels disclosed in Caliari S R, Burdick J A. A practical guide to hydrogels for cell culture. Nat Methods. 2016; 13(5):405-414, the disclosure of which is incorporated herein by reference in its entirety.

The hydrogel can be administered by injection. For example, the immunotherapy delivery hydrogel system can be administered using microneedles. The microneedles can include arrays of micro-projections generally ranging from about 25 μm to about 2000 μm in height. Microneedles can pierce the surface of the tissue to which they are applied, e.g., skin, to overcome its barrier, and facilitate delivery of an active agent associated with the hydrogel into the tissue. Microneedles include solid microneedles, coated microneedles, and hollow microneedles. Microneedles include dissolving and degradable microneedles and phase transition microneedles.

In some embodiments, the microneedles include the immunotherapy delivery hydrogel system. In some embodiments, the microneedles are fabricated from one or more hydrogel-forming polymers. Non-limiting examples of suitable polymers include poly(vinyl alcohol), amylopectin, carboxymethylcellulose (CMC) chitosan, poly(hydroxyethylmethacrylate) (polyHEMA), poly(acrylic acid), and poly(caprolactone), or a Gantrez™-type polymer. Gantrez™-type polymers include poly(methylvinylether/maleic acid), esters thereof and similar, related, polymers (e.g., poly(methyl/vinyl ether/maleic anhydride). In some embodiments, the microneedles can be formed from the same hydrogel matrix, e.g., biocompatible polymeric or polysaccharide, with which one or more therapeutic agent(s) and/or cancer cell attractant(s) are associated. In other embodiments, the microneedles include a material which is different from the hydrogel matrices of the hydrogel compositions disclosed herein, e.g., a material that coats the hydrogel compositions disclosed herein. Examples of microneedle arrays suitable for the use with the hydrogel compositions disclosed herein include hydrogel arrays described in U.S. Pat. Nos. 9,549,746 and 9,320,878, the disclosures of each of which are incorporated herein by reference in its entirety.

In some embodiments, microneedle-based hydrogel compositions are suitable for transdermal delivery of therapeutic agents such as one or more chemokines and/or immune checkpoint inhibitors or for delivery to the areas of the brain or body where a resection cavity cannot be formed.

In some embodiments, the cancer is any cancer where extended release of a therapeutic agent can be advantageous. For example, the cancer can include locations where total removal of the tumor tissue by surgery is challenging. For example, the cancer can include brain cancer, sarcoma, head and neck cancer, prostate cancer, anal cancer, cervical cancer, breast cancer, or any combination hereof.

The following examples are presented for the purpose of illustrating, not limiting the disclosure.

### EXAMPLES

### Example 1. Preparation of Hydrolysable Hydrogels and Critical Gel Point Determination

Four sets of hydrogel matrix master-mixes were generated: 4 mM PEG tetra-BCN (PEGtBCN) hydrogel precursor with a cleavable 4-azido diazide crosslinker or cleavable 2-azido diazide crosslinker and 3 mM precursor with a cleavable 4-azido diazide crosslinker or cleavable 2-azido diazide crosslinker. Each precursor was reacted with Alexafluor 568-azide derivative (AF568, 50 μM final concentration) to covalently conjugate the dye to the BCN groups present in the hydrogel matrix via the azide group. The resulting four hydrogels were then mixed with their respective crosslinker and 10 μL complete solutions were cast in Eppendorf tubes for 1 hour. PBS (200 μL) was added on top of the now-cast gels as the release media, and the reaction was allowed to incubate for a week. Each day, 5 μL supernatant samples of the hydrogel supernatant were collected, diluted with 50 μL PBS, and analyzed on a plate reader to quantify free-floating AF568 as a measurement of the hydrogel breakdown.

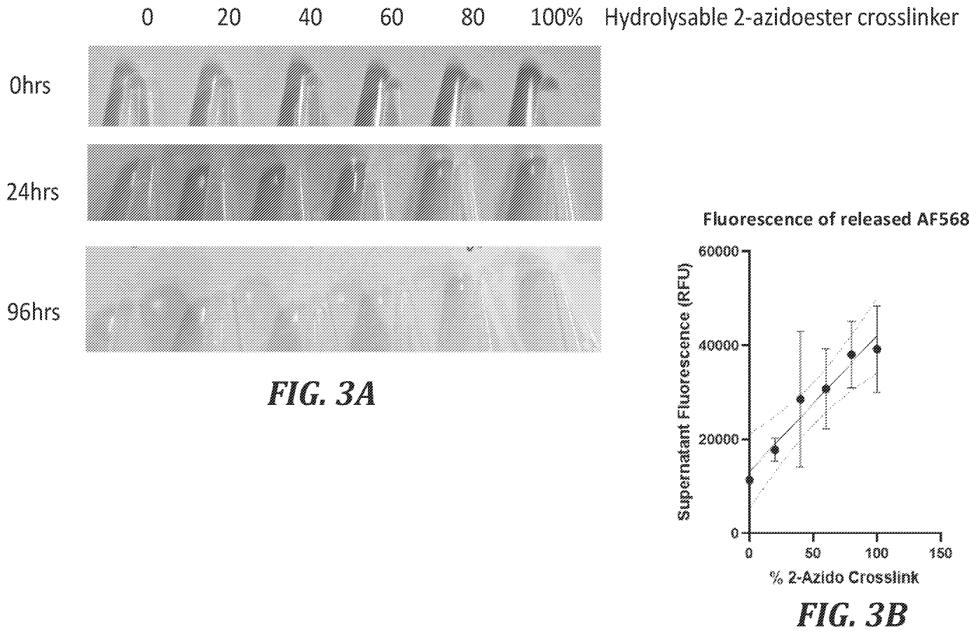

Referring to FIG. 3A, to determine the maximum number of crosslinks that could be broken before the hydrogel would break down completely, the following experiment was performed. Utilizing only the 4 mM PEG gels, 5 sets of 10 μL hydrogel variants were generated that were formulated with 0-100% hydrolysable 2-azido PEG-diazide crosslinker, with the remaining percentage of crosslinker (up to 100) replaced by non-hydrolysable 4-azido diazide crosslinker (a peptide amidated with 4-azido n-butanoic acid on the amino groups at the N-terminus and the side chain of the C-terminal lysine). Alexa Fluor 568 azide (AF568-zide, 50 μM) was conjugated to the backbone of each gel before polymerization as described above, and 200 μL PBS was added on top as release media. Samples were taken at 24-hour time points, up to 5 days, to record macroscopic gel degradation. At the conclusion of the experiment, the release media was read on a plate reader to quantify the extent of gel breakdown using the same dilution as described above. The structures of the components used in this experiment are shown below.

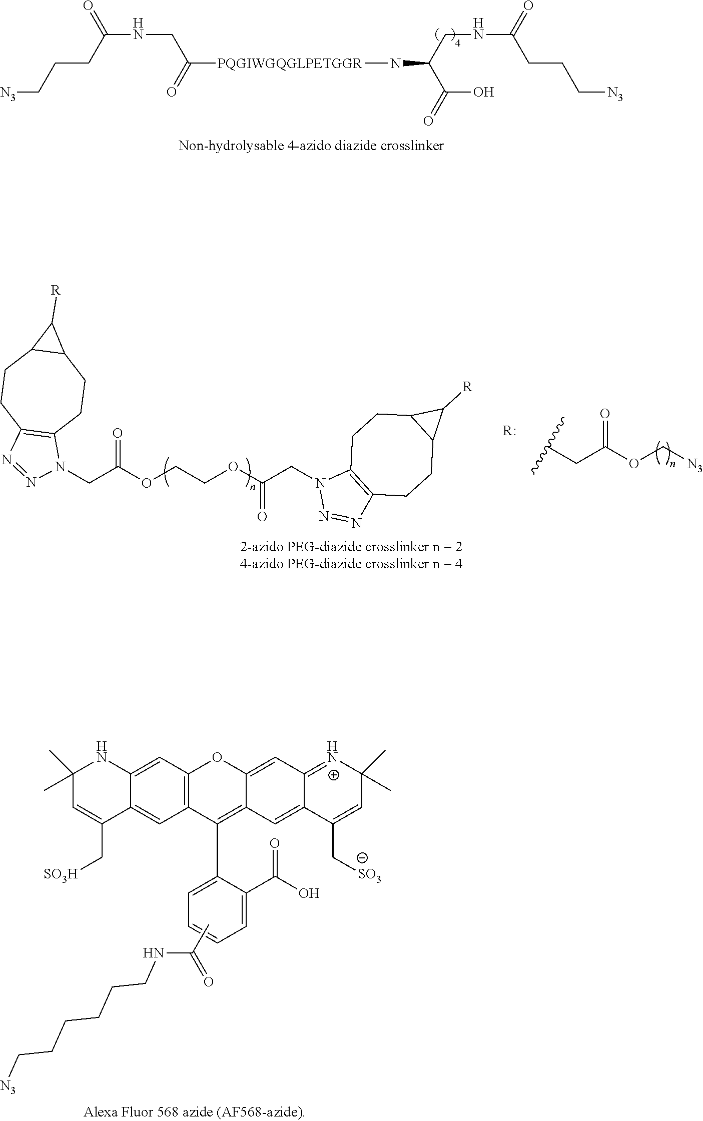

The hydrolysable gels degraded at a pre-determined rate based on the length of the azidoacid. AlexaFluor 568 was directly conjugated to the gel backbone and the fully cast gels were incubated in PBS for 96 hrs. The fluorescence of the supernatant was recorded to detect free-floating AF568 due to gel breakdown. Referring to FIG. 3B, regardless of PEG concentration, both sets of 2-azido gels reached maximum RFU of about 40,000 into the supernatant by days 2-3. The 4-azido gels, regardless of PEG concentration, demonstrated mild burst release initially that subsided quickly. This could be caused by some untethered AF568 that did not conjugate into the backbone. These gels did not release any appreciable amount of AF568 into the supernatant for the remainder of the experiment, suggesting their structural integrity hadn't broken down yet. A standard curve was generated using dilutions of AF568 and the raw RFU values were transformed into percentage released of AF568 into the supernatant. The 2-carbon azidoacid groups reached 100% release by 96 hours, while the 4-carbon variants did not change from its initial burst release of about 30%.

An experiment was carried out to define the critical gel point, or the amount of PEG-di-azide crosslinkers that could hydrolyze before the entire gel lost integrity and dissolved into PBS. PEGtBCN gels with AF568 attached to the backbone were cast using the 2-carbon hydrolysable crosslinker. The percentage of hydrolysable to unhydrolyzable crosslinker was varied from 0 to 100% to determine its critical gel point. Gels were cast with the indicated hydrolysable crosslinker percentage and left to incubate in PBS. Referring to FIG. 3A, by 24 hours, gels containing 60% or less hydrolysable crosslinker showed little macroscopic degradation while those with 80%+ were degraded. By 96 hours, gels containing 40% or less hydrolysable linker remained intact. It is believed that the gel can lose up to 40% of its crosslinks before completely dissolving.

The time course of release of AF568 from the gel was also measured. The supernatant of the dissolved gels in PBS was recorded on a plate reader after 96 hours to quantify the extend of gel breakdown by detecting the free floating AF568. Gels formulated with 0-40% hydrolysable crosslinker did not reach a maximum RFU of about 40,000 during this time point; however, those with 60-100% hydrolysable crosslinker were very close to the maximum.

### Example 2 Enhancing Local Delivery of Macrophage Checkpoint Inhibitors with Chemokine Gradients to Lure Migratory Pediatric Brain Tumor Cells

This Example investigates whether simultaneous delivery of chemokines and macrophage checkpoint inhibitors from a slow-release hydrogel depot within an infiltrative xenograft pHGG tumor can create a “trap” capable of enhanced phagocytic elimination of tumor cells. The data confirms gradients of classical immune cell chemokines, like CXCL12, are effective at eliciting chemotaxis of patient-derived pediatric high grade glioma (pHGG) tumor cells in vitro. CD47 mAb blockade and subsequent opsonization was an effective single agent to promote tumor cell elimination in co-cultures of murine and human macrophages in vitro. Intratumoral delivery of chemokines and CD47mAbs together from slow-release hydrogels within mouse brains demonstrated greater attenuated xenograph tumor growth based on bioluminescence data compared to antibody delivery alone. These results suggest macrophage checkpoint blockade could be enhanced by chemokine gradients to safely promote recruitment and immunological clearance of remnant brain tumor cells in young patients with incompletely resected brain tumors.

Variable Hydrolysis Rates of Payloads Conjugated to Azidoacids

If hydrogel-based delivery of therapeutics into the brain is to be attempted, it should have extended release capabilities so multiple surgeries can be avoided. Slow release also helps attenuate toxicities associated with burst release of drug into tissue. Release rates of payloads from hydrogel drug depots can be altered in a number of ways. Changing the size of the pores formed between polymer crosslinks or adjusting crosslink density itself can alter the diffusion characteristics of the hydrogel. Physical degradation of the polymer to release drug encapsulated in the polymer is a strategy employed by the Gliadel wafers. However, as those polymers degrade, it physically interfaces with a smaller surface of tissue, impacting its drug distribution profile. One strategy to achieve long term release rates from polymers while maintaining its structural integrity is to tether a given therapeutic payload to the backbone of the gel via cleavable linkers. Further taking advantage of SPAAC click chemistry, a type of azide-functionalized fatty acid (azidoacids) as either permanent or transient linkers between a therapeutic payload and the PEG gel can be used.

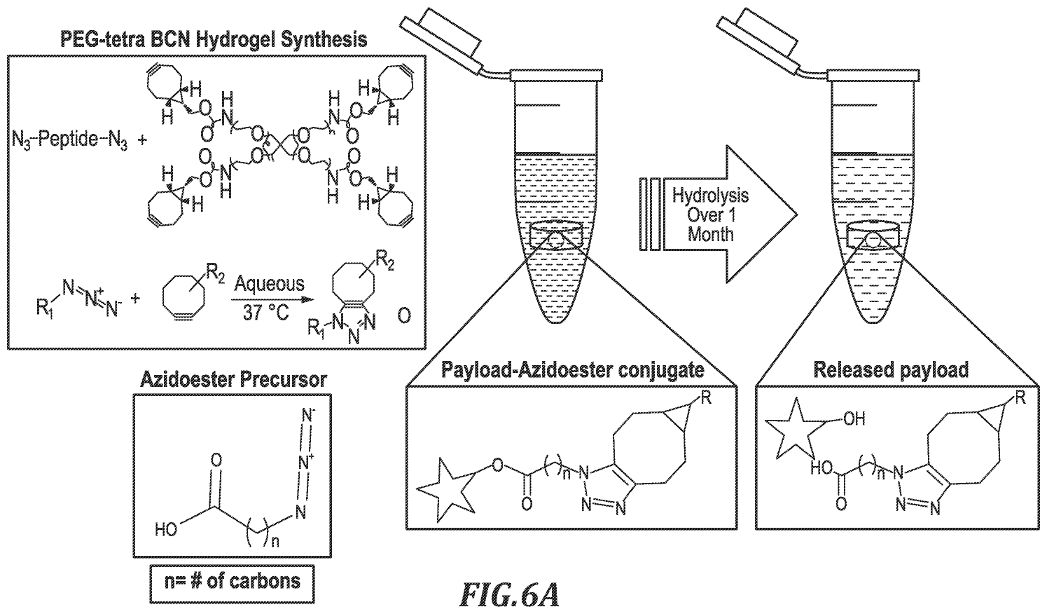

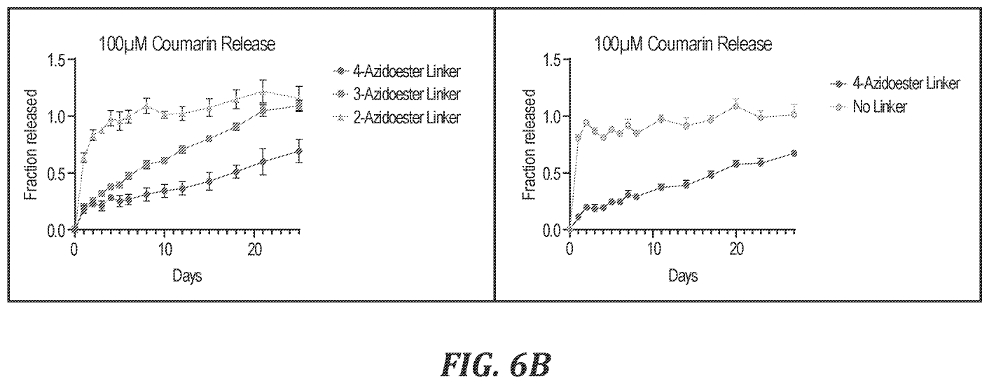

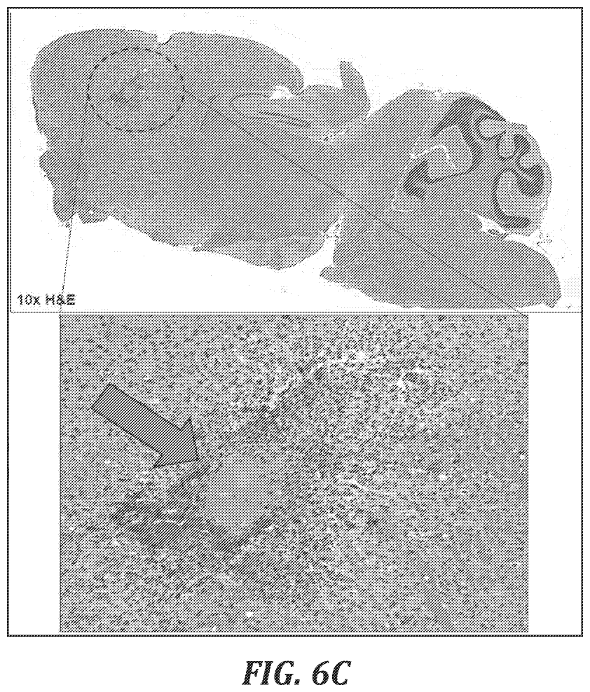

The azidoacid linkers can covalently attach to the alkynes of a PEG-BCN gel and, depending on the functional groups present, can form either an ester or amide with the payload. Esters formed between a payload and these linkers hydrolyze in an aqueous solution and release an attached payload at a rate determined by the length of the acid's carbon chain (FIG. 6B). This can bestow a degree of tunability to this release system not possible with unlinked drugs, particularly if multiple are to be used at once. Alternatively, molecules conjugated to the linker as an amide can permanently linked to the gel without the assistance of an enzyme or the use of non-physiological pH conditions.

Sortagging—Tailoring Release Rates of Therapeutic Payloads to Hydrogel Depots

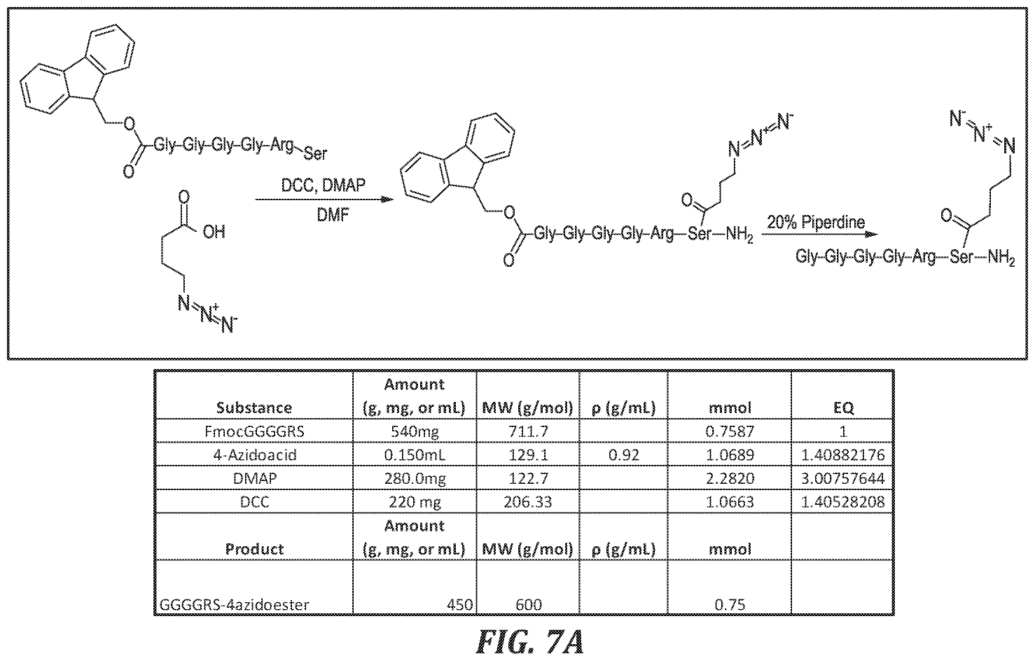

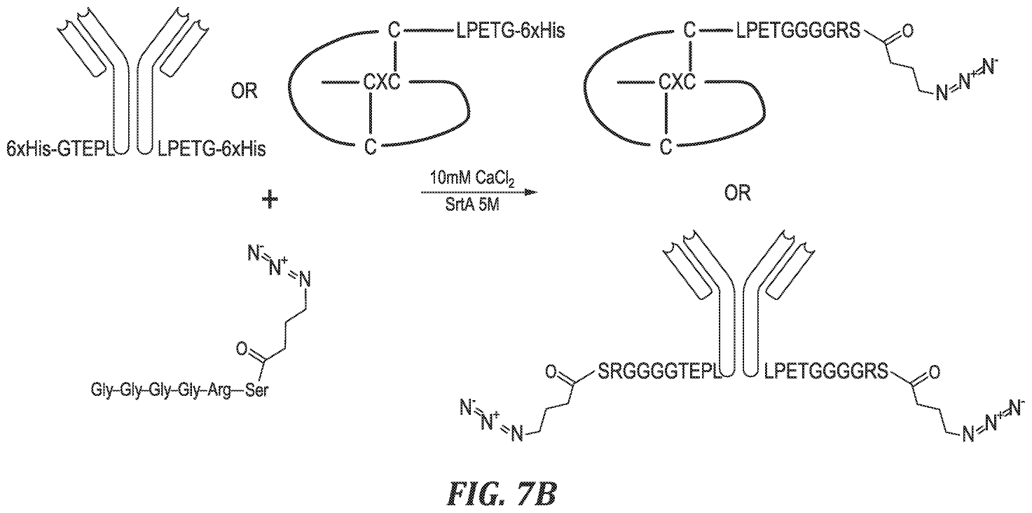

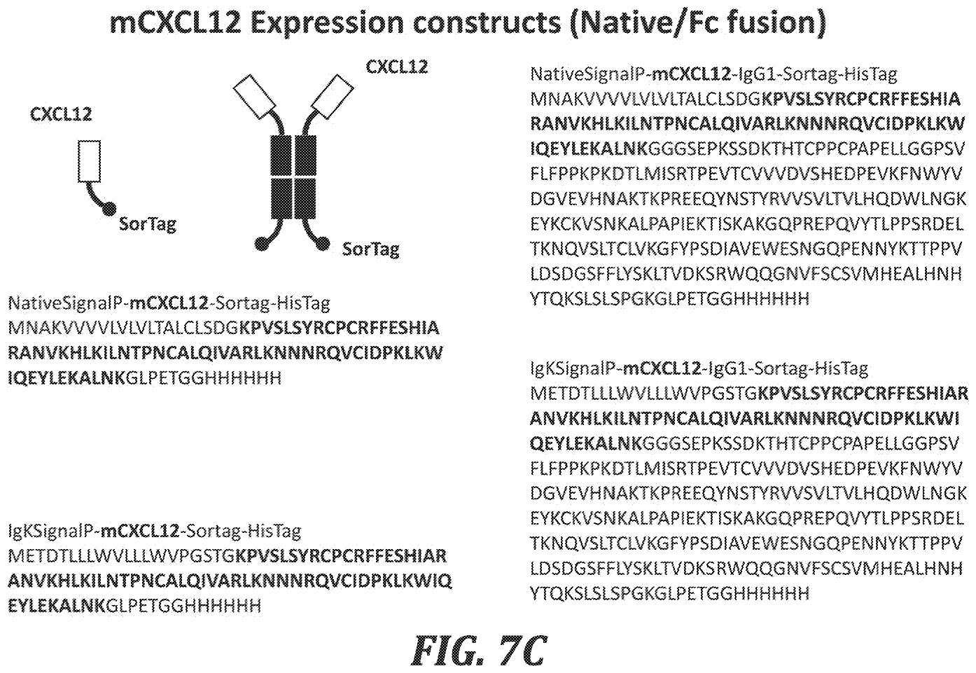

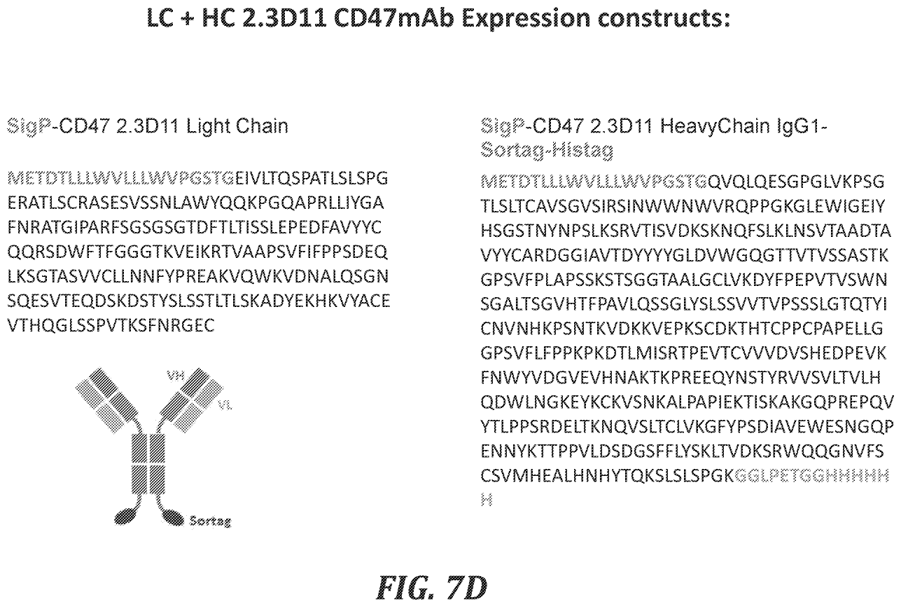

Referring to FIG. 7B, the heavy chain of an exemplary CD47mAb (SEQ ID NO:1) was modified at the C-terminus by incorporation of triglycine (GGG, a mock linker) using Sortase-mediated tagging. A 500 μL overnight reaction or 3 hours at 40° C., was set up using 20 μM CD47mAb-sortag, 2 mM GGG, 5 mM CaCl₂ and 1 μM of Sortase 5M enzyme. A negative control was set up in the same way, but without Sortase 5M. At the conclusion of the reactions, Cobalt-Agarose beads were incubated for 1 hr with both sets of the reactions to deplete any unreacted His-tagged antibody and Sortase. The supernatants containing antibody-GGG conjugates were collected, and elution buffer (20 mM Tris, 50 mM NaCl, 250 mM Imidazole) was added to the remaining beads to collect any bound His-tagged proteins. Addition of GGG to the antibody and loss of G-HHHHHH were confirmed by a western blot and SDS-PAGE.

Large proteins may have multiple serine residues, threonine residues, and tyrosine residues, whose free hydroxyl groups can form esters with a cleavable azidoacid linker, resulting in a stochastic release rate of a payload from the gel. Alternatively, or in addition, the primary amines at the N-terminus or lysines can generate amide bonds with an azidoacid, permanently linking a payload to the gel. In the interest of maintaining control of a payload-to-linker ratio and biological activity, there exists a simple enzymatic method of site-specifically modifying a particular biomolecule of interest with these azidoacids. Using the process called “sortagging” one may use the bacterial transpeptidase, sortase, to achieve this goal. By recombinantly expressing a biomolecule with a N- or C-terminal sortase recognition sequences, such as “LPXTG”, the sortase enzyme will conjugate triglycine (GGG)-containing molecules to this tag (FIG. 7B). Utilizing this strategy, a wide variety of molecular tags, including azidoacids, can be conjugated to a specific terminus of a biomolecule, resulting in a method of bridging an immunotherapeutic biomolecule and a PEG-BCN hydrogel via cleavable linkers to tunably control its release over time within the body.

Chemotaxis for Use Against Pediatric Brain Tumors

It is believed that a superior method of luring brain tumor cells into a therapeutic trap employ chemokine gradients to act on both tumor cells in direct contact and a distance away from a localized therapeutic source.

Unlike chemotherapy, antibodies, and other cytotoxic molecules, chemokines have activity at extremely low concentrations over long range in a gradient fashion. Furthermore, unlike a bundle of fibrous tracks, a therapeutic depot secreting chemokine gradients through the brain may not have to make direct contact with a bulky tumor, nor disseminated tumor cells in the parenchyma to attract them to the implant. It is believed that if such a chemokine lure system could be combined with a cytotoxic mechanism, then chemokine gradients can lure migratory tumor cells into a localized therapeutic source.

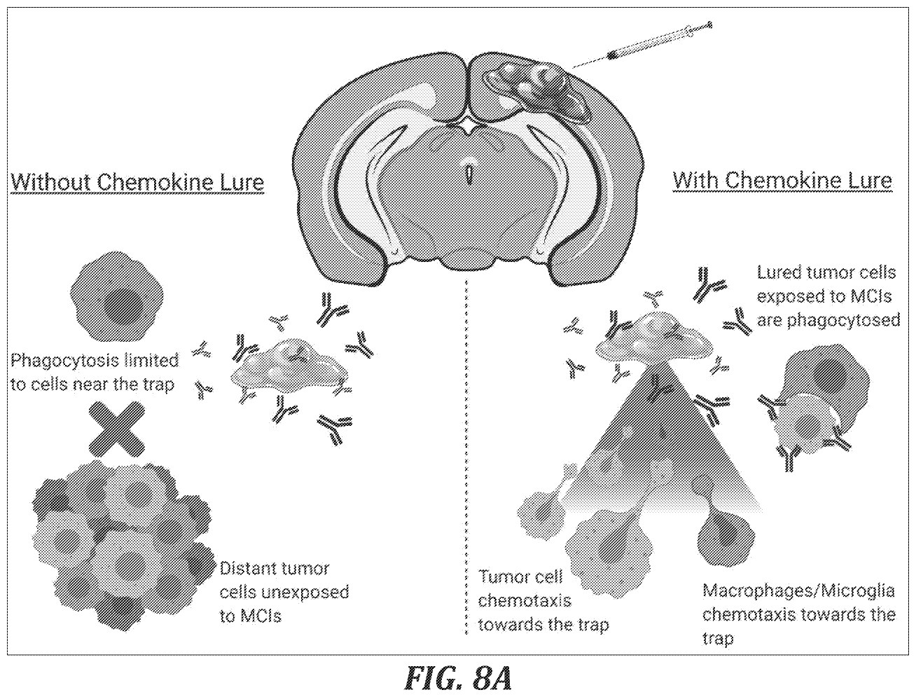

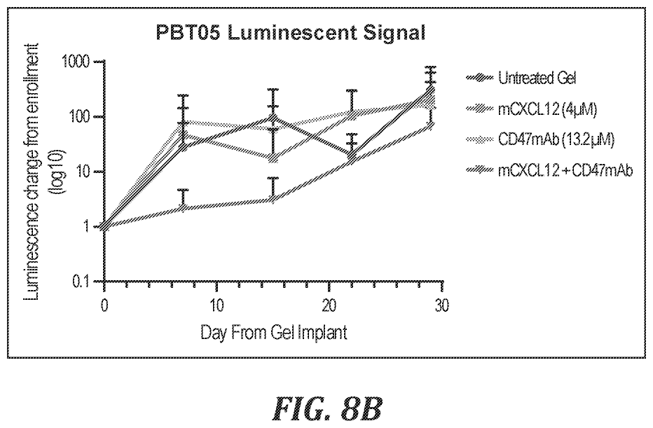

Given the shared sensitivity of immune cells and tumor cells to the same chemokine gradients, the incorporation of potent chemokines into a biomaterial platform loaded with immune cell-enhancing payloads would be favorable for cancer treatment. There can be a therapeutic window where potent chemokines, at low enough concentrations, can recruit tumor cells toward a localized immunotherapeutic “trap” while minimizing any boosts to their proliferation. It can be possible that a clinician could tailor the types of immune cells present in the microenvironment surrounding the implant, thus increasing the likelihood of successful treatment and the generation of an abscopal effect. Within the context of the PBTs, combining chemokine gradients with macrophage checkpoint inhibitors can enhance the effective cytotoxic range of an implanted material as a safe adjuvant treatment to eliminate remnant PBT cells. (FIG. 8A)

Results

Classical Chemokines Elicit Migration of pHGG Cells In Vitro

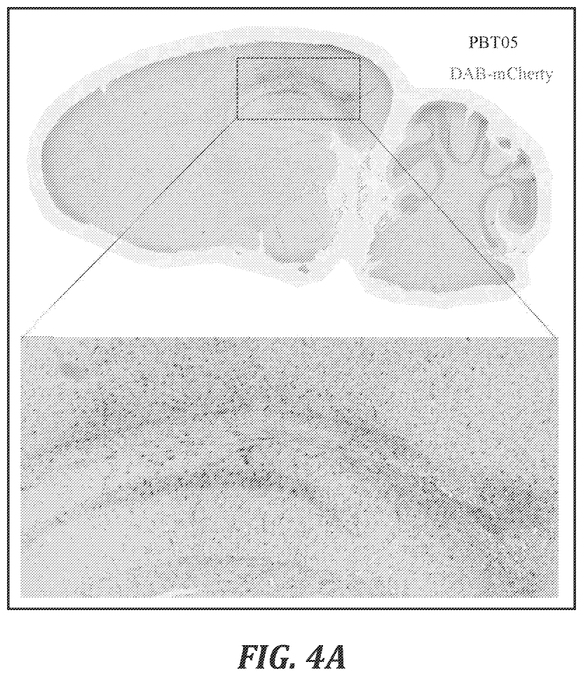

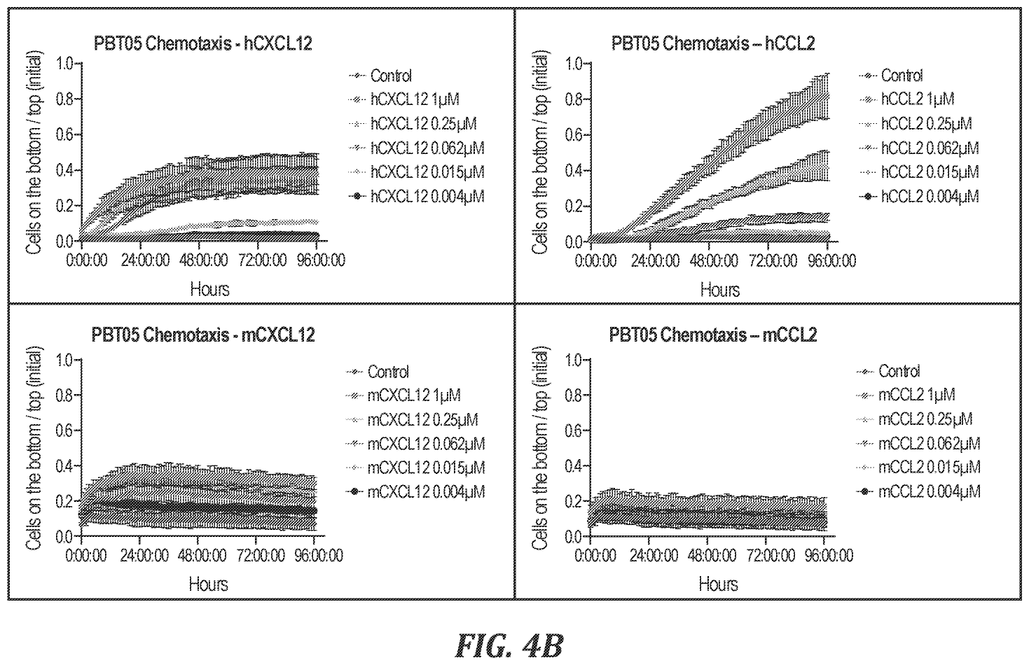

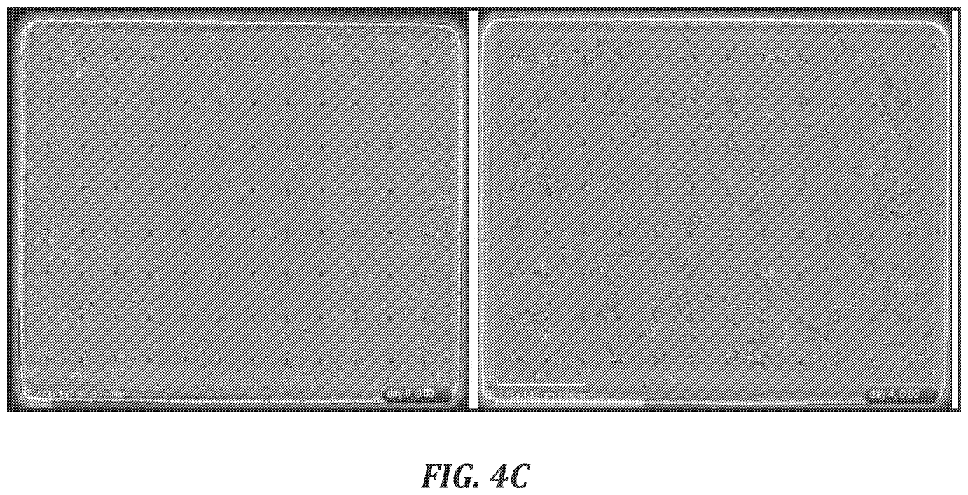

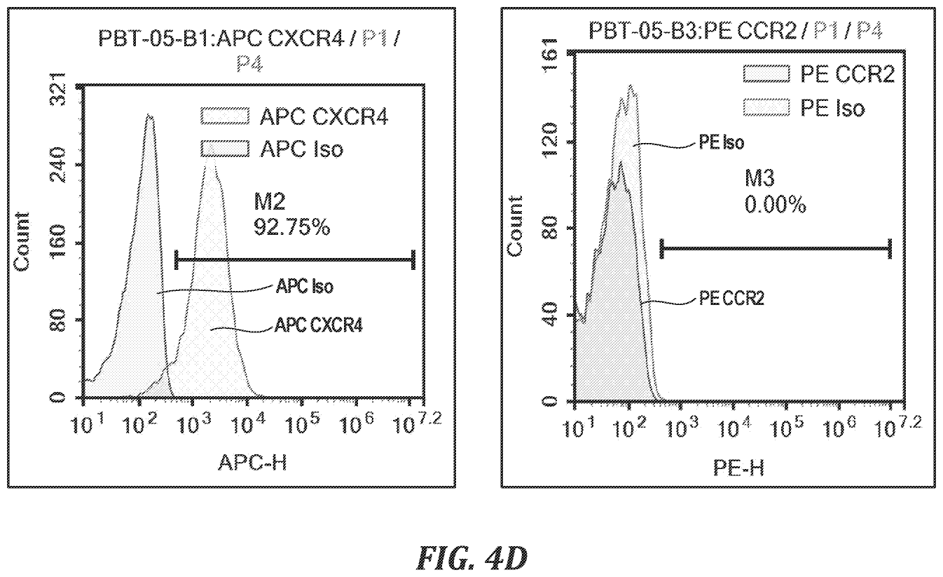

Locally delivered therapeutics in the brain may need to account for the tendency of remnant brain tumor cells to migrate away from the tumor margins—potentially out of reach locally delivered therapeutics. Here, artificial gradients of chemokines were used to lure tumor cells and phagocytic cells in the brain towards a static “trap” of macrophage-enhancing therapeutics to facilitate tumor cell elimination. Referring to FIGS. 4A-4D, to model this, a patient-derived, pediatric high grade glioma (pHGG) line, PBT-05, was used. This line demonstrates a similar infiltrative phenotype when engrafted into the brains of immunodeficient mouse models, as it did in the patient it is derived from. In vitro chemotaxis assays were carried out with PBT-05 against a panel of classical chemokines cited in literature as potentially being potent against migratory brain tumor cells. The results demonstrate that CCL2 and CXCL12 induced varying degrees of chemotaxis in PBT-05 cells. Human CCL2 (hCCL2) had the greatest potency of the chemokines tested, but PBT-05 was unresponsive to the murine version of this chemokine (mCCL2). Both human and murine CXCL12 (hCXCL12 and mCXCL12, respectively) had similar chemotactic potencies against PBT-05, though mCXCL12 lost some potency. Flow analysis of PBT-05 reveals no expression of CCR2, the canonical receptor for CCL2, but very high expression of CXCR4, the canonical receptor of CXCL12. Without wishing to be bound by theory, it is believed that PBT-05's attraction towards hCCL2 gradients may be mediated by another receptor promiscuous with this chemokine. While this receptor was not identified, mCCL2 may not bind to it or produce the same downstream signaling. However, its chemotactic sensitivity to human and murine CXCL12 may be mediated by the canonical pathway.

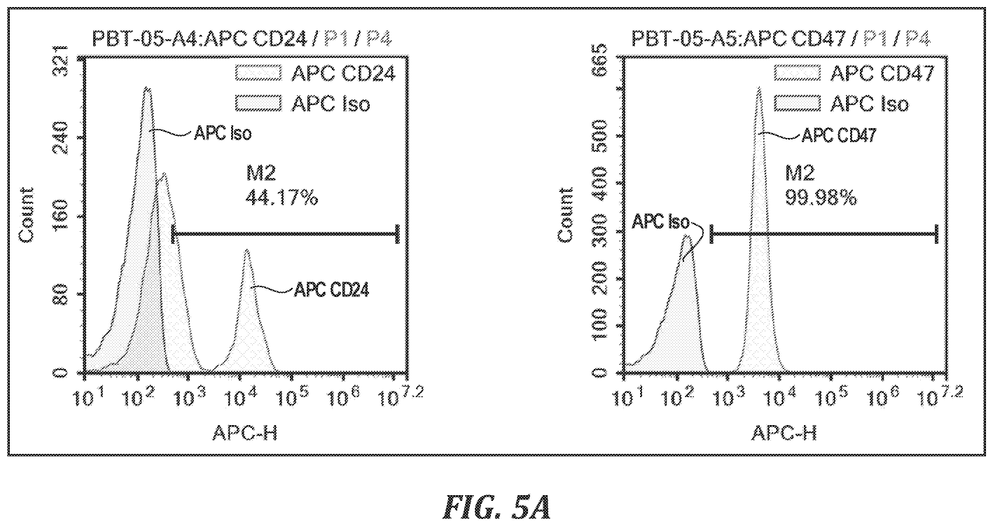

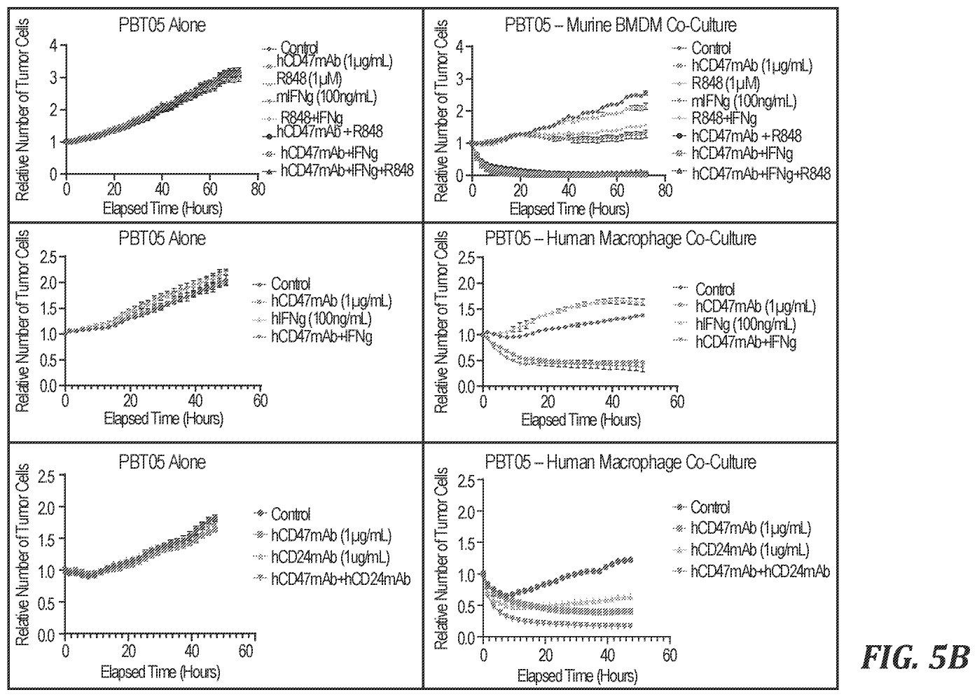

Monoclonal Antibody Blockade of Macrophage Checkpoints Promotes the Elimination of pHGG Cells by Human and Murine Macrophages In Vitro (FIGS. 5A-5B)

To determine the most potent factors to induce elimination of any lured brain tumor cells by macrophages and microglia near the immunotherapy “trap”, co-culture assays of PBT-05 cells with either murine bone marrow derived macrophages (BMDMs) or human macrophages derived from peripheral blood monocytes (PBMCs) were conducted. These cultures were challenged with combinations of macrophage checkpoint inhibitors (MCIs) and immune cell polarizing factors described by literature as capable of promoting an immune response against tumor cells. Flow analysis of PBT-05 line shows high expression of the macrophage checkpoint, CD47, and moderate levels of CD24, another macrophage checkpoint signal. Blockade of CD47 with as little as 1 ug/mL of monoclonal antibody was the most effective single agent at eliciting phagocytosis of PBT-05 cells by murine and human macrophages, although the effect was nearly maximized with murine macrophages. Inclusion of the polarizing agents, R848 and/or IFNg, did not significantly contribute to tumor cell cytotoxicity over CD47 blockade alone in murine macrophage co-cultures. However, combinatorial blockade of CD47 and CD24 showed greater phagocytosis of human tumor cells in human macrophage co-cultures than either antibody alone. Compared to a media control, no combination of MCIs and polarizing factors caused significant cytotoxicity of PBT-05 cells cultured alone, confirming the observed cytotoxic effect is mediated by the macrophages, not the drugs themselves.

Engineering User-Programmable Hydrogel Depots for Long Term Molecule Release In Vivo (FIGS. 6A-6C)

Local delivery of therapeutics into the brain should have long-lasting release capabilities to avoid repeat surgeries. To this end, PEG-tetraBCN hydrogels were engineered to act as an implantable “trap” capable of extended release of biomolecules like mCXCL12 and CD47mAbs within the brain's parenchyma. PEG chains elicit very little immune activity—necessary for reducing inflammation in the brain—and the arms of the chains can be modified with a variety of functional handles to bestow customizable properties to an otherwise-inert polymer. Conjugating therapeutic payloads to the PEG-tBCN backbone via hydrolysable azidoester linkers affords tunable, prolonged molecule release compared to diffusion alone. The programmability of this linker system in the gels was demonstrated by sustaining the release of 100 uM of a small fluorescent molecule, coumarin in vitro. Time until complete release of this molecule determined by the length of the azidoacids linker it was conjugated to. 100% release was achieved within 5 days using the 2-carbon linker, 21 days for the 3-carbon linker, and at the conclusion of this 28-day study, only 69% was released using the 4-carbon linker. The 4-carbon azidoester linker was chosen going forward due to its long-acting capabilities compared to diffusion alone. The SPAAC reaction involved in the polymerization of our gels can be slowed by chilling the solution to 4 degrees Celsius. With the reaction rate slowed, this complete gel solution was loaded into a pre-silanized Hamilton syringe. Using the Hamilton needle, a lateral incision was created in the parenchyma at the tumor's location and 2 μL of the still-liquid hydrogel solution was injected into this cavity. The mouse's body temperature facilitated the polymerization process in situ.

Generation of “Sortaggable” mCXCL12 and CD47mAb Variants for Extended Release (FIGS. 7A-7D)

Recombinant biomolecules of interest, like CXCL12 and CD47mAbs, can be site-specifically conjugated to the azidoester linkers for extended hydrogel release via sortase-mediated conjugation. Sortase recognizes “LPETG” sites on proteins of interest and can be used to conjugate GGG-containing molecular tags to the N- or C-termini of a protein. mCXCL12 were recombinantly expressed with LPETG sites at the C-terminus and “sortagged” it with a GGGGRS polypeptide that was pre-esterified to the 4-carbon azidoacid at its serine. “Sortaggable” variants of the 2.3D111 clone of the CD47mAb were also recombinantly expressed with LPETG recognition sites at both of its heavy chain C-termini and subsequently conjugated them onto our GGGRS-azidoester linkers. These “sortaggable” variants maintained biological activity as the store-bought variety and linking them to the PEG-tetraBCN gels demonstrated similarly prolonged release rates as coumarin in vitro.

FIG. 7C shows the sequences of MCXCL12 expression constructs:

(SEQ. ID NO. 4)
MNAKVVVVLVLVLTALCLSDGKPVSLSYRCPCRFF
ESHIARANVKHLKILNTPNCALQIVARLKNNNRQV
CIDPKLKWIQEYLEKALNKGLPETGGHHHHHH;
(SEQ. ID NO. 5)
METDTLLLWVLLLWVPGSTGKPVSLSYRCPCRFFE
SHIARANVKHLKILNTPNCALQIVARLKNNNRQVC
IDPKLKWIQEYLEKALNKGLPETGGHHHHHH;
(SEQ. ID NO. 6)
MNAKVVVVLVLVLTALCLSDGKPVSLSYRCPCRFF
ESHIARANVKHLKILNTPNCALQIVARLKNNNRQV
CIDPKLKWIQEYLEKALNKGGGSEPKSSDKTHTCP
PCPAPELLGGPSVFLFPPKPKDTLMISRTPEVTCV
VVDVSHEDPEVKFNWYVDGVEVHNAKTKPREEQYN
STYRVVSVLTVLHQDWLNGKEYKCKVSNKALPAPI
EKTISKAKGQPREPQVYTLPPSRDELTKNQVSLTC
LVKGFYPSDIAVEWESNGQPENNYKTTPPVLDSDG
SFFLYSKLTVDKSRWQQGNVFSCSVMHEALHNHYT
QKSLSLSPGKGLPETGGHHHHHH;
and
(SEQ. ID NO. 7)
METDTLLLWVLLLWVPGSTGKPVSLSYRCPCRFFE
SHIARANVKHLKILNTPNCALQIVARLKNNNRQVC
IDPKLKWIQEYLEKALNKGGGSEPKSSDKTHTCPP
CPAPELLGGPSVFLFPPKPKDTLMISRTPEVTCVV
VDVSHEDPEVKFNWYVDGVEVHNAKTKPREEQYNS
TYRVVSVLTVLHQDWLNGKEYKCKVSNKALPAPIE
KTISKAKGQPREPQVYTLPPSRDELTKNQVSLTCL
VKGFYPSDIAVEWESNGQPENNYKTTPPVLDSDGS
FFLYSKLTVDKSRWQQGNVFSCSVMHEALHNHYTQ
KSLSLSPGKGLPETGGHHHHHH.

FIG. 7D shows the sequences for the light chain and heavy chain CD47mAb expression constructs:

(SEQ. ID NO. 8)
METDTLLLWVLLLWVPGSTGEIVLTQSPATLSLSP
GERATLSCRASESVSSNLAWYQQKPGQAPRLLIYG
AFNRATGIPARFSGSGSGTDFTLTISSLEPEDFAV
YYCQQRSDWFTFGGGTKVEIKRTVAAPSVFIFPPS
DEQLKSGTASVVCLLNNFYPREAKVQWKVDNALQS
GNSQESVTEQDSKDSTYSLSSTLTLSKADYEKHKV
YACEVTHQGLSSPVTKSFNRGEC;
and
(SEQ ID NO. 2)
METDTLLLWVLLLWVPGSTGQVQLQESGPGLVKPS
GTLSLTCAVSGVSIRSINWWNWVRQPPGKGLEWIG
EIYHSGSTNYNPSLKSRVTISVDKSKNQFSLKLNS
VTAADTAVYYCARDGGIAVTDYYYYGLDVWGQGTT
VTVSSASTKGPSVFPLAPSSKSTSGGTAALGCLVK
DYFPEPVTVSWNSGALTSGVHTFPAVLQSSGLYSL
SSWTVPSSSLGTQTYICNVNHKPSNTKVDKKVEPK
SCDKTHTCPPCPAPELLGGPSVFLFPPKPKDTLMI
SRTPEVTCVVVDVSHEDPEVKFNWYVDGVEVHNAK
TKPREEQYNSTYRVVSVLTVLHQDWLNGKEYKCKV
SNKALPAPIEKTISKAKGQPREPQVYTLPPSRDEL
TKNQVSLTCLVKGFYPSDIAVEWESNGQPENNYKT
TPPVLDSDGSFFLYSKLTVDKSRWQQGNVFSCSVM
HEALHNHYTQKSLSLSPGKGGLPETGGHHHHHH.

Intratumoral Release of CD47mAb and CXCL12 Promotes Prolonged Attenuation of pHGG Xenograft Growth In Vivo (FIGS. 8A-8B)

To characterize the tumoricidal capability of co-delivered CD47mAb and mCXCL12, untreated gels, gels with linked and unlinked CD47mAb, gels with linked mCXCL12 or a combination of the two were intratumorally injected within the brains of athymic Nu−/Nu− mice harboring Luciferase-expressing PBT-05 tumors. These mice are deficient in B, T, and NK cells to allow for tumor cell engraftment, but have a fully intact innate immune system which allows the study in phagocytic cell activity alone. The gels were implanted for 4 weeks and bioluminescence time course data showed steady growth in tumors receiving untreated gels, CXCL12-containing gels, and CD47mAb antibody-alone gels. In contrast, the group of mice receiving gels that contained the combination of CD47mAb+mCXCL12 resulted in arrested tumor growth relative to the other treatments for just over two weeks before the effect began tapering off.

Discussion

Complete elimination of pediatric brain tumor cells is fraught with challenges. Surgical resection of tumors in certain locations of the brain carries the risk of causing long-lasting neurological deficits in the patient. In many cases, surgeons know that it is necessary to leave microscopic or macroscopic disease behind. In this proof-of-concept demonstration, hydrogel-based delivery of chemokines and blocking antibodies to macrophage checkpoint ligands on the tumor cell surface shows potential to be a viable strategy for safely luring pediatric brain tumor cells into an immunotherapy “trap” for their elimination by phagocytic cells within the brain. While the in vivo model demonstrated positive results when delivering these factors into fully-intact bulky tumors, this strategy is believed to be most effective at eliminating residual disease cells in patients after much of the tumor was debulked.

Delivery of any soluble factors that stimulate the immune system must be carefully controlled, particularly in the brain. Cytokines and CD47 mAbs have been shown clinically to elicit systemic toxicity on their own. Delivery of these factors from slow-release depots would allow a surgeon to safely deliver higher concentrations of therapeutics than would be possible with systemic drug administration. To this end, PEG-based hydrogels were engineered to act as a simultaneous in vivo delivery system of chemoattractants and MCIs. PEG is inherently low in toxicity, and the click chemistry involved in the polymerization of these gels allows for either permanent or labile coupling of cytokines and other therapeutic agents to the gel backbone in a cytocompatible manner. To demonstrate the tunability of this system for releasing therapeutics, variable release rates of small molecules as well as biomolecules like CXCL12 were shown. Gels coupled to these payloads on hydrolysable linkers demonstrated release profiles spanning a few days to beyond an entire month. This flexibility could allow controlled release of a cocktail of molecules at different rates, if needed. As demonstrated, recombinantly expressed biomolecules can be C-terminally ligated to these linkers via sortase tagging and maintain their biological function. Thus, this system can be adapted for a wide variety of uses, not limited to chemokines and antibodies.

Antibody-mediated phagocytosis of tumor cells relies on both tumor and immune cell types residing in close proximity to each other. Some high-grade brain tumor cells are known to migrate away from the tumor cavity, potentially further than the reach of locally administered mAbs. This was discovered to limit the efficacy of localized chemotherapy, like the FDA approved GLIADEL, to eliminate residual brain tumor cells. Without wishing to be bound by theory, it is believed that this problem could be overcome if nearby brain tumor cells could be coaxed closer to the therapeutic source. Some tumor cells express receptors for chemokines physiologically produced in metastatic niches such as the leptomeningeal space of the brain. Although some therapeutic strategies seek to block chemokine receptors on tumor cells to restrict their movement, here this behavior was used to bring brain tumor cells closer to immune cells and immunotherapeutic agents. That gradients of classical immune cell chemokines such as CXCL12 was found to be effective at eliciting migration of brain tumor cells in vitro. Assuming a biopsy of the brain tumor could be obtained to determine chemokine receptor expression, a variety of chemokines could be employed for this purpose to control for potency as well as mitogenicity on a case-by-case basis. This technique has flexibility and potential to become a universal method to attract migratory tumor cells out of nearby, unreachable locations of the brain without causing additional physical disruption to the tissue to gain access to them.

Monoclonal antibody blockade of the cell-surface “don't eat me” ligand, CD47, was the most effective single agent at eliciting tumor cell elimination by both human and murine macrophages, even at low concentrations of antibody. The differences in magnitude of the CD47mAb effect between murine and human macrophages suggests there may be multiple “don't eat me” axes besides CD47-SIRPa impeding the phagocytic activity of human macrophages. Indeed, the monoclonal antibody blockade of CD47 and CD24, another macrophage checkpoint, afforded greater tumor cell destruction by human macrophages than either antibody alone. Furthermore, the macrophages and microglia within the brain may not need to be polarized for maximum tumor cell consumption when using these antibodies. R848 and IFNg, which induce activated phenotypes in macrophages, had mild effects on the viability of tumor cells in co-culture and did not significantly increase cytotoxicity when combined with CD47mAbs. This may be clinically relevant because activated macrophages can non-selectively damage cells around them via toxic NO-release, a consideration while working within the space of very sensitive nervous tissue.

In summary, a novel therapeutic combination and delivery strategy to eliminate pediatric brain tumor cells was created. Classical immune cell chemokines, like CXCL12, effectively chemoattract a pediatric HGG brain tumor line. This approach synergizes with methods of antibody-mediated opsonization and blockade of macrophage checkpoint signals, like CD47, that are often over-expressed on the surface of tumor cells. Delivering these factors directly into the brain via an implantable, slow release hydrogel recruited both immune cells and brain tumor cells into a tumoricidal environment that would have otherwise not favored immunological engagement. The data not only demonstrate the safety and efficacy of this combinatorial approach when used in mice, but also highlights the sheer customizability of this system for a variety of uses. The modularity of the PEG-tetraBCN hydrogel chemistry affords clinicians the flexibility to mix and match therapeutic payloads, optimize their concentrations, and fine tune their release rates to extend the effect were observed. Overall, these results demonstrate the feasibility of combining chemokines, MCIs, and other forms of immunomodulatory factors to create a tumor cell “trap” within tissue. This Example can be generalized to a wide variety of cancers given the fact that chemokine receptors are expressed on many cancer types.

### Materials and Methods

Animal and Tumor Cell Line Preparation

Patient-derived xenograft (PDX) cells were obtained from autopsy or biopsy (SCH/COG). PDX lines were cultured in Neuralcult NS-A Basal Medium (Stem Cell) with Proliferation supplement (Stem Cell, 05753), PenStrep (Thermofisher), Glutamax (Thermofisher) EGF (Peperotech, AF-100-15) and FGF (Peperotech, 100-18B). Cells were grown adherent on tissue-culture treated plates after at least 2 hours of Laminin coating (Sigma-Aldrich) in an incubator at 37° C. in 5% CO2. All PDX lines were lentivirally transduced with H2b-GFP, mCherry and Luciferase to assist in cell counting and tumor size visualization via IVIS. Xenograft tumors were established in the cortex of female Athymic Nu−/Nu− (Harlan) mice (age). Tumors were allowed to grow to z flux value of 1e⁶ before study enrollment. All mouse studies were carried out following protocols approved by the IACUC at FHCRC (protocol 1457) and complied with all relevant ethical regulations.

In Vitro Chemotaxis Assays

Chemokines were purchased from: Sigma-Aldrich (CCL2: SRP3109) and RnD Systems (CXCL12: 350-NS/CF). Cell migration assays were performed using the chemotaxis module on the Incucyte Zoom 2016 and S3 (Essen Bio). Specialized 96 well transwell plates and reservoir dishes were supplied by the manufacturer (cat no's 4582, 4600). H2B-GFP+ PBT05 lines cells were cultured in the Neuralcult media+supplement without EGF/FGF to reduce background mobility caused by these growth factors. The Incucyte software was tailored to identify GFP+ nuclei to count the number of tumor cells on top and bottom of the transwell membranes.

Isolation of Murine Macrophages from Mouse Bone Marrow and Human Macrophages from PBMCs

Murine monocytes were harvested and cultured from femurs of C57BL/6 mice using RPMI (Thermofisher, 11875093) containing 10% heat deactivated FBS and 100 ng/mL mCSF1 for 7 days. Mature macrophages from these cultures were later harvested for experiments. Human monocytes were isolated from human PBMCs (Bloodworks) and purified by EasySep monocyte depletion kit (StemCell, 19355). Monocytes were cultured in RPMI containing 10% heat deactivated FBS and 25 ng/mL human CSF1 for 10 days. 50 ng/mL IL-10 was added to the cultures at day 5 to induce M2 phenotypes. Mature macrophages were later harvested for use in experiments.

In Vitro Phagocytosis Assays

Monoclonal antibodies were purchased from Bioxcell (LEAF hCD47, BE0019) and Biolegend (LEAF hCD24mAb: 101810, MsIgG1: 400153). Phagocytosis assays were performed using the Basic Analyzer software on Essen Bio's Incucyte Zoom and Incucyte S3. 12 or 24 well plates were seeded 1:1 with GFP+ tumor lines and macrophages (murine/human) in fully supplemented Neuralcult plus various MCIs and immunomodulators described previously. Using the aforementioned definitions, the Incucyte calculated tumor cell counts based on the number of GFP+ nuclei in the wells over time.

Release of Coumarin-1-OH from PEG-tetraBCN Hydrogels

Coumarin-1-OH was synthesized in house and conjugated to 2-azidoacetic acid (Click chemistry tools, 1081) 3-azidopropionic acid (Click chemistry tools, 1082) and 4-azidobutyric acid (made in house) using Steglich esterification in DCM. Conjugates were then incorporated into the backbone of PEG-tetraBCN hydrogels and 25 μL gels were plated in triplicate into a 12 well plate containing 500 uL PBS/well. The plate was left in an incubator at 37 degrees Celsius 5% CO₂. Supernatants were taken from each well according to the experimental timepoints, read on a plate reader for fluorescence (Molecular Devices VersaMax) and added back to each well. Fluorescence of released coumarin was related to concentration using linear regression of a coumarin standard in PBS.

Synthesis of GGGGRS-4Azidoesters

Fmoc-GGGGRS, was synthesized using the Liberty peptide synthesizer (CEM) with amino acids purchased from ChemImpex (Glycine: 02416, Arginine: 01964, Serine: 02454). The peptide was then HPLC purified in 70/30 H₂0/Acetonitrile and lyophilized. 1.06 mmol 4-Azidobutyric acid (Synthonix, A1941), 0.75 mmol Fmoc-GGGGRS, 2.28 mmol DMAP (Sigma-Aldrich, 8.51055) and 1.06 mmol DCC (Sigma-Aldrich, D80002) were stirred for 3 hours at 40 degrees Celsius in minimal DMF (Sigma-Aldrich, 319937). Upon completion, the reaction was Fmoc-deprotected with the addition of Piperidine (ChemImpex, 02351) to a final concentration of 20%, stirring for 1 minute. The peptide was then precipitated in di-ethyl ether, HPLC purified using 95/5 H₂0/Acetonitrile and lyophilized. Purified GGGGRS-4Azidoesters was stored at −20 Celsius for later use under a nitrogen atmosphere.

Expression of “Sortaggable” mCXCL12 and 2.3D11 CD47mAbs

The mature amino acid sequence for mCXCL12 was obtained from NCBI, GeneID 20315. The VH and VL sequences for the 2.3D11 CD47mAb was obtained from U.S. Pat. No. 9,650,441 B2. Gblocks (IDT) were created for these sequences, modified with “LPETG” sortase recognition sites and a 6×his tag at the C-terminus of mCXCL12 and the VH C-terminus of the 2.3D11 CD47mAb. Gblocks containing these sequences were ligated into double digested expression plasmids using Gibson assembly (NEB). Assembled plasmids were transformed into chemically competent STBL3 E. coli (Thermofisher) and sequenced for accuracy. Plasmids were subsequently maxiprepped (Qiagen, 12162) and transduced into HEK293F for protein expression. Supernatants containing the molecules of interest were collected and purified via Ni-NTA pulldown and AKTA FPLC (Cytiva).

Conjugation of mCXCL12 and 2.3D11 CD47mAb to GGGGRS-4Azidoesters Purified chemokines and antibodies were buffer exchanged into sortase reaction buffer (50 mM Tris, 125 mM NaCl, 10 mM CaCl2) pH 7.5). 50 μM mCXCL12 or CD47mAb was reacted with 2.5 μM Sortase 5M and 1 mM GGGRS-4azidoester in 500p SrtA reaction buffer for 2 hr at 37 degrees Celsius in a shaking incubator. 500 μL of Ni-NTA (Thermofisher, 88221) beads were added to the completed reaction for 1 hour at 4 degrees Celsius to sequester any un-reacted antibody, chemokine and Sortase 5M. The reaction was spun at 10 k G's for 2 minutes and the supernatant containing the linked chemokine/antibody conjugates was removed and buffer exchanged back into PBS for downstream use.

Release of mCXCL12-4Azidoester and CD47-4Azidoester from PEG-tetraBCN Hydrogels

mCXCL12-4azidoesters and CD47-4azidoesters were conjugated overnight to the backbone of PEG-tetraBCN hydrogels and 25 μL gels were arranged into 4 groups of triplicates and plated into 12-well dishes containing 500 uL PBS/well. Plates were stored in an incubator at 37 C 5% CO₂. At the conclusion of each week, the PBS supernatants from each corresponding group (week 1, week 2, etc.) were collected and stored at −80 C until the conclusion of the experiment. Supernatants were later thawed and analyzed using a BCA assay (Pierce) to assess the protein concentration in each supernatant at the conclusion of each week.

Bioluminescence Imaging

PBT05 lines were transduced by lentivirus to express a cytoplasmic Luciferase-mCherry construct. Mice harboring Luc+ tumors were injected with D-Luciferin (Xenolight) at concentrations of 3 mg/100 uL PBS per mouse. 3 minutes post injection of D-Luciferin, mice were anesthetized using isoflurane for an additional 7 minutes. 10 minutes post Luciferin injection, anesthetized mice were placed in the IVIS (Perkin Elmer) chamber and bioluminescence imaging was obtained with 1 minute exposure time, F/stop 1 and 8, field D. Luminescent photos and total flux ROIs were analyzed using Living Image software (PerkinElmer).

IHC and Tissue Imaging

Xenograft mouse brains were harvested, formalin fixed, and paraffin embedded. Brain block were then sliced and stained for DAB-GFP and DAB-F4/80. IHC sections were imaged using a TISSUEFAX slide scanner (Gnosis) in the imaging core at FHCRC.

Preparation and Cortical Injection of PEG-Tetra BCN Hydrogel Solutions into Athymic Nu−/Nu− Mice Bearing H2bGFP+/Luciferase+/mCherry+ Tumors

80 uL hydrogel master mixes were created for each treatment group at a final concentration of 3.25 mM (6.5%) PEG-tetraBCN (20 kDa) and 6.5 mM PEG-diazide (3.5 kDa). For treatment groups containing mCXCL12-4azidoester and/or CD47-4azidoester, solutions of PEG-tBCN and the payloads were pre-reacted at 37 degrees Celsius for 2 hours in a shaking incubator to have time for backbone incorporation before crosslinker addition. PEG-diazide and PEGtBCN master mixes (+/− mCXCL12-4azido, +/−CD47mAb-4azido) were placed on ice in separate tubes. When ready to be used, both tubes were combined, vortexed, and brought up to a final volume of 80 uL in PBS and placed back on ice to slow the polymerization rate of the now-forming gel network. 3 uL of complete hydrogel master mix was quickly loaded into a pre-silanized 10μL Hamilton Neuros syringe (Hamilton, #65460-05) and injected cortically into isoflurane-anesthetized mice. The needle stop was set to 2 mm of depth and the still-liquid master mix was administered into a cavity created by the lateral movement of the needle within the brain. Gels were targeted to the same location in the brain as the original tumor implant, as indicated by a depression in the skull from the initial implant surgery.

By example and without limitation, embodiments are disclosed according to the following enumerated paragraphs:

A1. An immunotherapy delivery hydrogel system, comprising:

a hydrogel matrix;

a tumor cell-attractant conjugated to the hydrogel matrix; and

a cancer therapeutic agent associated with the hydrogel matrix;

wherein the tumor cell-attractant and the cancer therapeutic agent are synergistic in treating cancer and are controllably released from the immunotherapy delivery hydrogel system.

A2. The immunotherapy delivery hydrogel system of Paragraph A1, wherein the hydrogel matrix is cleavable.

A3. The immunotherapy delivery hydrogel system of Paragraph A1 or Paragraph A2, wherein the hydrogel matrix comprises a crosslinked poly(ethylene glycol).

A4. The immunotherapy delivery hydrogel system of any one of Paragraphs A1 to A3, wherein the hydrogel matrix comprises an ester linkage, an amide linkage, a disulfide linkage, an acetal linkage, a ketal linkage, an oxime linkage, a hydrazone linkage, or any combination thereof.

A5. The immunotherapy delivery hydrogel system of any one of Paragraphs A1 to A4, wherein the hydrogel matrix comprises a crosslinking moiety of Formula (III):

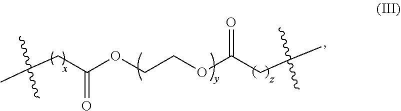

wherein:

x and z are each independently an integer selected from 1, 2, 3, 4, 5, and 6, and

y is an integer of from 1 to 50.

A6. The immunotherapy delivery hydrogel system of any one of Paragraphs A1 to A5, wherein the tumor cell-attractant, the cancer therapeutic agent, or both, are each released from the immunotherapy delivery hydrogel system at a predetermined rate.

A7. The immunotherapy delivery hydrogel system of any one of Paragraphs A1 to A6, wherein the tumor cell-attractant, the cancer therapeutic agent, or both, are covalently bound to the hydrogel matrix.

A8. The immunotherapy delivery hydrogel system of any one of Paragraphs A1 to A7, wherein the tumor cell-attractant, the cancer therapeutic agent, or both, are hydrolytically or enzymatically cleavable from the hydrogel matrix.

A9. The immunotherapy delivery hydrogel system of any one of Paragraphs A1 to A8, wherein the tumor cell-attractant, the cancer therapeutic agent, or both, are physically entrapped in the hydrogel matrix.

A10. The immunotherapy delivery hydrogel system of any one of Paragraphs A1 to A9, wherein the tumor cell-attractant, the cancer therapeutic agent, or both, are associated with the hydrogel matrix via non-covalent interactions.

A11. The immunotherapy delivery hydrogel system of any one of Paragraphs A1 to A10, wherein the cancer cell attractant comprises a chemokine, a cytokine, an anti-cancer therapeutic agent, an immune stimulatory agent, or a combination thereof.

A12. The immunotherapy delivery hydrogel system of Paragraph A11, wherein the chemokine is selected from mCXCL12, CXCL12, CCL2, mCCL2, CX3CL1, mCX3CL1, and any combination thereof.

A13. The immunotherapy delivery hydrogel system of any one of Paragraphs A1 to A12, wherein the therapeutic agent comprises an antibody or a binding fragment thereof, an immune stimulatory molecule, a bispecific T-cell engager, an immune checkpoint molecule, an immune cell (e.g., modified T-cells and/or NK cells), or any combination thereof.

A14. The immunotherapy delivery hydrogel system of Paragraph A13, wherein the antibody or a binding fragment thereof is an immune checkpoint inhibitor, an anti-CD47 antibody, an anti-CD24 antibody, an anti-PD-L1 antibody, an anti-B7H3 antibody, or a binding fragment thereof, or any combination thereof.

A15. The immunotherapy delivery hydrogel system of Paragraph A14, wherein the anti-CD47 antibody or a binding fragment thereof comprises a sequence having at least 90% homology to SEQ ID NO: 1.

A16. The immunotherapy delivery hydrogel system of Paragraph A14 or Paragraph A15, wherein the anti-CD47 antibody or a binding fragment thereof comprises an enzyme recognition sequence at the C-terminus.

A17. The immunotherapy delivery hydrogel system of any one of Paragraphs A14 to A16, wherein the anti-CD47 antibody or a binding fragment thereof comprises a sortase recognition sequence at the C-terminus.

A18. The immunotherapy delivery hydrogel system of Paragraph A13, wherein the immune stimulatory molecule comprises IFNg or IL-4, IL-2, IL-15, a fusion of IL-15_IL-15 Receptor Alpha, or any combination thereof.

A19. The immunotherapy delivery hydrogel system of Paragraph A13, wherein the immune checkpoint molecule comprises PD-L1, CTLA-4, CD47, CD24, CD155, CD112, β2 Microglobulin (B2M), or any combination thereof.

A20. The immunotherapy delivery hydrogel system of Paragraph A13, wherein the immune cell is entrapped in the hydrogel matrix.

A21. The immunotherapy delivery hydrogel system of any one of Paragraphs A1 to A13, comprising a hydrogel matrix; a chemokine conjugated to the hydrogel matrix; and a macrophage checkpoint antibody or a fragment thereof associated with the hydrogel matrix.

A22. The immunotherapy delivery hydrogel system of any one of Paragraphs A1 to A21, wherein the rate of release of the therapeutic agent from the immunotherapy delivery hydrogel system is greater than the rate of degradation of the hydrogel matrix, in a biological environment.

A23. A method of treating cancer, comprising:

administrating the immunotherapy delivery hydrogel system of any one of Paragraphs A1 to A22 to a subject in need thereof,

wherein the cancer comprises an upregulation of PD-L1, CTLA-4, CD47, CD24, CD155, CD112, β₂ Microglobulin (B2M), or any combination thereof.

A24. The method of Paragraph A23, wherein the immunotherapy delivery hydrogel system is at a temperature lower than the body temperature of the subject during administration.

A25. The method of Paragraph A23, wherein the immunotherapy delivery hydrogel system is at a temperature higher than the body temperature of the subject during administration.

A26. The method of any one of Paragraphs A23 to A25, comprising administrating the immunotherapy delivery hydrogel system to a tumor tissue.

A27. The method of any one of Paragraphs A23 to A26, wherein administration of the immunotherapy delivery hydrogel system to the subject occurs after surgical removal of a tumor or a portion of the tumor.

A28. The method of any one of Paragraphs A23 to A27, wherein the immunotherapy delivery hydrogel system is administered into a tissue directly abutting a cavity formed by surgical removal of a tumor or a portion of the tumor.

A29. The method of any one of Paragraphs A23 to A28, wherein the immunotherapy delivery hydrogel system releases at least one of the cancer cell attractant and the cancer therapeutic agent.

A30. The method of any one of Paragraph A23 to A29, wherein the immunotherapy delivery hydrogel system simultaneously releases the cancer cell attractant and the cancer therapeutic agent.

A31. The method of any one of Paragraphs A23 to A30, wherein the immunotherapy delivery hydrogel system releases a cancer cell attractant and a cancer therapeutic agent, and the cancer therapeutic agent attracts macrophages and/or microglia.

A32. The method of any one of Paragraphs A23 to A31, wherein the cancer is selected from brain cancer, sarcoma, head and neck cancer, prostate cancer, anal cancer, cervical cancer, breast cancer, or any combination hereof.

A33. A method of making a recombinant protein, comprising:

providing a gene fragment for a protein sequence comprising an enzyme-recognizable label;

inserting the gene fragment into a plasmid;

transducing the plasmid into a mammalian cell;

expressing a protein encoded by the gene fragment; and isolating the protein.

A34. A method of Paragraph A33, wherein the protein is a cytokine or an antibody comprising an enzyme-recognizable label at a C-terminus.

A35. A method of Paragraph A33 or Paragraph A34, wherein the enzyme recognizable label is a sortase-recognizable label.

A36. A method of any one of Paragraphs A33 to A35, further comprising reacting the protein in the presence of an enzyme recognizing the enzyme-recognizable label, a compound comprising a chemically reactive group recognizable by the enzyme, to provide a protein conjugated to the compound comprising the chemically reactive group.

A37. A method of Paragraph A36, wherein the chemically reactive group comprises SH, OH, amino, COOH, an activated ester, N₃, optionally substituted maleimide, optionally substituted heteroaryl (e.g., tetrazine), optionally substituted C₃-C₆ alkenyl, ethynyl, optionally substituted C₃-C₆ alkynyl, or an optionally substituted C₈-C₁₂ cycloalkynyl reactive groups.

A38. A method of any one of Paragraphs A33 to A37, wherein the mammalian cell is a HEK293T cell.

While illustrative embodiments have been illustrated and described, it will be appreciated that various changes can be made therein without departing from the spirit and scope of the disclosure.

1. An immunotherapy delivery hydrogel system, comprising:
a hydrogel matrix;
a tumor cell-attractant conjugated to the hydrogel matrix; and a
cancer therapeutic agent associated with the hydrogel matrix;
wherein the tumor cell-attractant and the cancer therapeutic agent are synergistic m treating cancer and are controllably released from the immunotherapy delivery hydrogel system.

2. The immunotherapy delivery hydrogel system of claim 1, wherein the hydrogel matrix is cleavable.

3. The immunotherapy delivery hydrogel system of claim 1, wherein the hydrogel matrix comprises a crosslinked poly(ethylene glycol).

4. The immunotherapy delivery hydrogel system of claim 1, wherein the hydrogel matrix comprises an ester linkage, an amide linkage, a disulfide linkage, an acetal linkage, a ketal linkage, an oxime linkage, a hydrazone linkage, or any combination thereof.

5. The immunotherapy delivery hydrogel system of claim 1, wherein the hydrogel matrix comprises a crosslinking moiety of Formula (III): 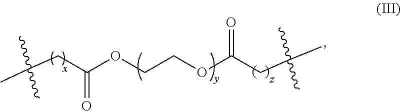
wherein:
x and z are each independently an integer selected from 1, 2, 3, 4, 5, and 6, and y is an integer of from 1 to 50.

6. The immunotherapy delivery hydrogel system of claim 1, wherein the tumor cell-attractant, the cancer therapeutic agent, or both, are each released from the immunotherapy delivery hydrogel system at a predetermined rate.

7. The immunotherapy delivery hydrogel system of claim 1, wherein the tumor cell-attractant, the cancer therapeutic agent, or both, are covalently bound to the hydrogel matrix.

8. The immunotherapy delivery hydrogel system of claim 1, wherein the tumor cell-attractant, the cancer therapeutic agent, or both, are hydrolytically or enzymatically cleavable from the hydrogel matrix.

9. The immunotherapy delivery hydrogel system of claim 1, wherein the tumor cell-attractant, the cancer therapeutic agent, or both, are physically entrapped in the hydrogel matrix.

10. The immunotherapy delivery hydrogel system of claim 1, wherein the tumor cell-attractant, the cancer therapeutic agent, or both, are associated with the hydrogel matrix via non-covalent interactions.

11. The immunotherapy delivery hydrogel system of claim 1, wherein the cancer cell attractant comprises a chemokine, a cytokine, an anti-cancer therapeutic agent, an immune stimulatory agent, or a combination thereof.

12. The immunotherapy delivery hydrogel system of claim 11, wherein the chemokine is selected from mCXCL12, CXCL12, CCL2, mCCL2, CX3CL1, mCX3CL1, and any combination thereof.

13. The immunotherapy delivery hydrogel system of claim 1, wherein the therapeutic agent comprises an antibody or a binding fragment thereof, an immune stimulatory molecule, a bispecific T-cell engager, an immune checkpoint molecule, an immune cell (e.g., modified T-cells and/or NK cells), or any combination thereof.

14. The immunotherapy delivery hydrogel system of claim 13, wherein the antibody or a binding fragment thereof is an immune checkpoint inhibitor, an anti-CD47 antibody, an anti-CD24 antibody, an anti-PD-LI antibody, an anti-B7H3 antibody, or a binding fragment thereof, or any combination thereof.

15. The immunotherapy delivery hydrogel system of claim 14, wherein the anti-CD47 antibody or a binding fragment thereof comprises a sequence having at least 90% homology to SEQ ID NO: 1.

16. The immunotherapy delivery hydrogel system of claim 15, wherein the anti-CD47 antibody or a binding fragment thereof comprises an enzyme recognition sequence at the C-terminus.

17. The immunotherapy delivery hydrogel system of claim 16, wherein the anti-CD47 antibody or a binding fragment thereof comprises a sortase recognition sequence at the C-terminus.

18. The immunotherapy delivery hydrogel system of claim 13, wherein the immune stimulatory molecule comprises IFNg or IL-4, IL-2, IL-15, a fusion of IL-15_IL-15 Receptor Alpha, or any combination thereof.

19. The immunotherapy delivery hydrogel system of claim 13, wherein the immune checkpoint molecule comprises PD-L1, CTLA-4, CD47, CD24, CD155, CDI 12, p2 Microglobulin (B2M), or any combination thereof.

20. The immunotherapy delivery hydrogel system of claim 13, wherein the immune cell is entrapped in the hydrogel matrix.

21. The immunotherapy delivery hydrogel system of claim 1, comprising a hydrogel matrix; a chemokine conjugated to the hydrogel matrix; and a macrophage checkpoint antibody or a fragment thereof associated with the hydrogel matrix.

22. The immunotherapy delivery hydrogel system of claim 1 or claim 2, wherein the rate of release of the therapeutic agent from the immunotherapy delivery hydrogel system is greater than the rate of degradation of the hydrogel matrix, in a biological environment.

23. A method of treating cancer, comprising:
administrating the immunotherapy delivery hydrogel system of any one of claim 1 to a subject in need thereof,
wherein the cancer comprises an upregulation of PD-L1, CTLA-4, CD47, CD24, CD155, CD112, p₂ Microglobulin (B2M), or any combination thereof.

24-32. (canceled)

33. A method of making a recombinant protein, comprising:
providing a gene fragment for a protein sequence comprising an enzyme-recognizable label;
inserting the gene fragment into a plasmid; transducing the plasmid into a mammalian cell;
expressing a protein encoded by the gene fragment; and isolating the protein.

34-38. (canceled)


SEQUENCE LISTING (10 sequences)

SEQ ID NO: 1 - protein, 475 aa, Artificial sequence
  Synthetic
METDTLLLWVLLLWVPGSTGQVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 489 aa, Artificial sequence
  Synthetic
METDTLLLWVLLLWVPGSTGQVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGLPETGGHHHHHH

SEQ ID NO: 3 - protein, 5 aa, Artificial sequence
  Synthetic
  misc_feature (3)..(3): Xaa can be any naturally occurring amino acid
LPXTG

SEQ ID NO: 4 - protein, 102 aa, Artificial sequence
  Synthetic
MNAKVVVVLVLVLTALCLSDGKPVSLSYRCPCRFFESHIARANVKHLKILNTPNCALQIVARLKNNNRQVCIDPKLKWIQEYLEKALNKGLPETGGHHHHHH

SEQ ID NO: 5 - protein, 101 aa, Artificial sequence
  Synthetic
METDTLLLWVLLLWVPGSTGKPVSLSYRCPCRFFESHIARANVKHLKILNTPNCALQIVARLKNNNRQVCIDPKLKWIQEYLEKALNKGLPETGGHHHHHH

SEQ ID NO: 6 - protein, 338 aa, Artificial sequence
  Synthetic
MNAKVVVVLVLVLTALCLSDGKPVSLSYRCPCRFFESHIARANVKHLKILNTPNCALQIVARLKNNNRQVCIDPKLKWIQEYLEKALNKGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGLPETGGHHHHHH

SEQ ID NO: 7 - protein, 337 aa, Artificial sequence
  Synthetic
METDTLLLWVLLLWVPGSTGKPVSLSYRCPCRFFESHIARANVKHLKILNTPNCALQIVARLKNNNRQVCIDPKLKWIQEYLEKALNKGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGLPETGGHHHHHH

SEQ ID NO: 8 - protein, 233 aa, Artificial sequence
  Synthetic
METDTLLLWVLLLWVPGSTGEIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 243 aa, Artificial Sequence
  Synthetic
KPVSLSYRCPCRFFESHIARANVKHLKILNTPNCALQIVARLKNNNRQVCIDPKLKWIQEYLEKALNKLELPETGGGSGGSGGSGGSGGSMAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVKHHHHHH

SEQ ID NO: 10 - protein, 81 aa, Artificial sequence
  Synthetic
KPVSLSYRCPCRFFESHIARANVKHLKILNTPNCALQIVARLKNNNRQVCIDPKLKWIQEYLEKALNKLELPETGHHHHHH